(19) United States
(12) Patent Application Publication
(10) Pub. No.: US 2022/0275364 A1
(43) Pub. Date: Sep. 1, 2022

(54) VIVO GUIDE RNA LIBRARIES AND METHODS OF MAKING THE SAME

(71) Applicant: Whitehead Institute for Biomedical Research, Cambridge, MA (US)

(72) Inventors: Kristin Knouse, Boston, MA (US); Heather R. Keys, Quincy, MA (US)

(21) Appl. No.: 17/588,038

(22) Filed: Jan. 28, 2022

Related U.S. Application Data
(60) Provisional application No. 63/143,761, filed on Jan. 29, 2021.

(51) Int. Cl. C12N 15/11; C12N 15/10; C12N 9/22; C12N 15/86; C12N 15/90; G01N 33/50; A61K 49/00

(52) U.S. Cl. CPC C12N 15/11; C12N 15/1065; C12N 9/22; C12N 15/86; C12N 15/907; G01N 33/5088; A61K 49/0004; C12N 2310/20; C12N 2800/80; C12N 2740/15043; C12N 2840/002; C12N 2320/11

(57) ABSTRACT

Disclosed herein are methods and non-human mammals for in vivo functional genomic screens.

### RELATED APPLICATIONS

This application claims the benefit of U.S. Provisional Application No. 63/143,761, filed on Jan. 29, 2021, the entire teachings of which are incorporated herein by reference.

### GOVERNMENT SUPPORT

This invention was made with government support under DP5OD026369 awarded by the National Institutes of Health. The government has certain rights in the invention.

### BACKGROUND OF THE INVENTION

A complete understanding of the genetic determinants underlying cell fitness and function in mammalian tissues is limited by the availability of tools for high-throughput genetic dissection in intact organisms. Genome-wide screening using CRISPR-Cas9 has emerged as a powerful approach for the comprehensive investigation of cellular phenotypes, but to reliably assess depletion as well as enrichment it requires stable delivery of a single guide RNA (sgRNA) library to tens of millions of cells at a low multiplicity of infection. Transposon-based methods can achieve genome-scale sgRNA library delivery in vivo, but the high number of integrations in each cell increases the probability of complex genomic rearrangements and complicates assignment of phenotype to genotype, particularly in the case of negative selection. Thus far, lentiviral delivery of genome-scale sgRNA libraries has only been achieved in cells ex vivo, limiting genome-wide CRISPR screening to cell culture systems or cellular transplantation models (Chen et al., Cell 160, 1246-1260 (2015); Dong et al., Cell 178, 1189-1204.e23 (2019)). These ex vivo systems cannot always reproduce the cellular phenotypes desired for study and, even when they can, they cannot recapitulate the entirety of extracellular factors that normally influence these phenotypes in vivo.

### SUMMARY OF THE INVENTION

Understanding of mammalian physiology and disease would greatly benefit from the ability to perform high-throughput functional genomics within a living organism. Genome-wide CRISPR screening has emerged as a powerful method for dissecting gene function, but the need to stably deliver sgRNAs to millions of cells has thus far precluded its implementation in vivo. Here, the inventors have overcome this obstacle to establish genome-wide CRISPR screening in the mouse liver and identify the genetic requirements for cell fitness within an animal The screen uncovers novel sex-specific and cell non-autonomous regulation of hepatocyte fitness, including an essential role for the class I major histocompatibility complex. This screening approach can be readily adapted to other contexts, enabling unprecedented insight into how genes regulate cellular phenomena within the organism.

Some aspects of the present disclosure are directed to a method for generating an in vivo library for genomic screening, comprising providing a non-human mammal comprising cells in an organ or tissue having a sequence (e.g., having a sequence integrated into the genome) encoding a Cas protein or a functional portion thereof, introducing a plurality of single guide RNAs (sgRNA) into the non-human mammal with a viral vector to obtain an in vivo library for genomic screening comprising cells of the tissue or organ, wherein each cell comprises a sequence encoding a single guide RNA of the plurality of single guide RNAs.

In some embodiments, the non-human mammal is a mouse or rat. In some embodiments, the non-human mammal is neonate or embryo. In some embodiments, the non-human mammal has a disease or condition, is predisposed to a disease or condition, or has a genetic abnormality In some embodiments, the organ or tissue is the liver or hepatic tissue. In some embodiments, the cells are proliferating.

In some embodiments, the Cas protein or functional fragment thereof is selected from the group consisting of Cas9, or a catalytically inactive Cas protein fused to an effector domain In some embodiments, the effector domain is selected from the group consisting of a transcription activation domain, transcriptional repression domain, chromatin organizer domain, a remodeler domain, a histone modifier domain, a DNA modification domain, an RNA binding domain, a protein interaction input devices domain, and a protein interaction output device domain In some embodiments, the Cas protein or functional fragment thereof comprises a detectable label. In some embodiments, expression of the Cas protein or functional fragment thereof is under control of an inducible promoter and/or wherein expression of the plurality of sgRNAs are under control of an inducible promoter.

In some embodiments, the plurality of sgRNA target each of the expressed genes of one or more pathways of the cells. In some embodiments, the plurality of sgRNA target the expressed genes of the cells or a substantial portion thereof. In some embodiments, an average of more than two sgRNA species target each expressed gene. In some embodiments, an average of five or more sgRNA species target each expressed gene. In some embodiments, the plurality of sgRNA target at least, 20, 50, 100, 500, 1000, 5000, or more expressed genes. In some embodiments, the plurality of sgRNA comprise one or more species targeting one or more control genes.

In some embodiments, the viral vector is introduced by injection into a vein. In some embodiments, the vein is a superficial vein. In some embodiments, the vein is a temporal vein.

In some embodiments, the viral vector integrates a nucleotide sequence encoding the sgRNA into the genome of the cells, optionally wherein the viral vector is a lentiviral or retroviral vector. In some embodiments, about 5*10⁷ transduction units of viral vector are introduced into the non-human mammal In some embodiments, about 10%, 20%, 25%, 50%, 75%, or 90% of the cells in an organ or tissue comprise sgRNA.

Some aspects of the present disclosure are directed to a non-human mammal comprising an in vivo library described herein or generated by the methods disclosed herein.

Some aspects of the present disclosure are directed to a method of in vivo screening for genomic sites in cells in an organ or tissue associated with a change in phenotype, comprising providing a non-human animal comprising an in vivo library of a population of cells in an organ or tissue having a sequence encoding a Cas protein or a functional portion thereof and a sequence encoding an sgRNA of a plurality of sgRNAs targeting the genomic sites, providing conditions under which the Cas protein or functional portion thereof and the sgRNA contact and modify the genomic sites, detecting one or more phenotypic changes in the population of cells and determining a genomic site associated with the phenotypic change by identifying a sgRNA associated with the phenotypic change and determining the sgRNA target genomic site.

In some embodiments, the genomic sites are expressed genes in the population of cells. In some embodiments, the phenotype is survival, proliferation, resistance or susceptibility to an agent, cell differentiation, morphology, expression of a gene or protein, protein localization, or resistance or susceptibility to a disease or condition. In some embodiments, the non-human mammal is a mouse or rat. In some embodiments, the non-human mammal is neonate or embryo. In some embodiments, the non-human mammal has a disease or condition, is predisposed to a disease or condition, has a genetic abnormality, or has been contacted with an agent. In some embodiments, the organ or tissue is the liver or hepatic tissue. In some embodiments, the population of cells is proliferating.

In some embodiments, the Cas protein or functional fragment thereof is selected from the group consisting of Cas9, or a catalytically inactive Cas protein fused to an effector domain. In some embodiments, wherein the effector domain is selected from the group consisting of a transcription activation domain, transcriptional repression domain, chromatin organizer domain, a remodeler domain, a histone modifier domain, a DNA modification domain, a RNA binding domain, a protein interaction input devices domain, and a protein interaction output device domain In some embodiments, the Cas protein or functional fragment thereof comprises a detectable label. In some embodiments, expression of the Cas protein or functional fragment thereof is under control of an inducible promoter.

In some embodiments, expression of the plurality of sgRNAs are under control of an inducible promoter. In some embodiments, the plurality of sgRNA target each of the expressed genes of one or more pathways of the population of cells. In some embodiments, the plurality of sgRNA target the expressed genes of the population of cells or a substantial portion thereof. In some embodiments, an average of more than two sgRNA species target each expressed gene. In some embodiments, an average of five or more sgRNA species target each expressed gene. In some embodiments, the plurality of sgRNA target at least, 20, 50, 100, 500, 1000, 5000, or more expressed genes. In some embodiments, the plurality of sgRNA comprise one or more species targeting one or more control genes.

In some embodiments, the conditions comprise inducing expression of the Cas protein or functional portion thereof and/or the plurality of sgRNAs. In some embodiments, the population of cells constitutively express the plurality of sgRNAs and wherein the conditions comprise inducing expression of the Cas protein or functional portion thereof. In some embodiments, the expression of the Cas protein or functional portion thereof is under control of a Cre-lox system.

In some embodiments, the period of time between the performance of providing conditions under which the Cas protein or functional portion thereof and the sgRNA contact and modify the genomic sites and detecting one or more phenotypic changes in the population of cells is sufficient to allow for one or more divisions of the cells of the population of cells.

In some embodiments, the step of detecting one or more phenotypic changes in the population of cells comprises harvesting the population of cells and detecting the relative abundance (e.g., enrichment and depletion) of each sgRNA species in the population of cells.

In some embodiments, the method further includes identifying, based on the relative abundance for each sgRNA, enrichment and/or depletion of genomic regions (e.g., expressed genes) in the population of cells. In some embodiments, the identified genomic regions are further assessed, e.g., in drug discovery screens, animal models of diseases, etc.

In some embodiments, about 10%, 20%, 25%, 50%, 75%, or 90% of cells in the organ or tissue comprise sgRNA.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1F. shows efficient delivery of sgRNAs and depletion of long-lived proteins in the liver. (FIG. 1A) Lentiviral vectors for U6-driven expression of an sgRNA and hepatocyte specific-expression of a fluorescent reporter (mCherry or mTurq2). (FIG. 1B) Images of endogenous mCherry and mTurq2 fluorescence in livers from mice four days after injection of an equal mixture of sgAAVS1-mCherry and sgAAVS1-mTurq2 lentiviruses. Livers were counterstained with phalloidin (green) to label actin. Scale bars, 100 μm. (FIG. 1C) Percent mCherry-, mTurq2-, and double-positive hepatocytes in livers from mice four days after injection with an equal mixture of sgAAVS1-mCherry and sgAAVS1-mTurq2 lentiviruses. n=3 mice per dose and 200 hepatocytes per mouse. Error bars indicate standard deviation. (FIG. 1D) Scheme for inducing protein depletion in LSL-Cas9 mice. (FIG. 1E) Images of livers from LSL-Cas9 mice injected with sgMaob-mCherry followed by PBS or AAV-Cre immunostained for mCherry (magenta), MAO-B (green), and actin (blue). Scale bars, 45 μm. (FIG. 1F) Cytoplasmic MAO-B intensity per μm in mCherry-positive and mCherry-negative hepatocytes from LSL-Cas9 mice injected with sgMaob-mCherry followed by PBS or AAV-Cre. n=1 male and 1 female mouse per condition and 25 cells per mouse. Closed and open circles represent values from male and female mouse, respectively.

FIGS. 2A-2G. A genome-wide screen for hepatocyte fitness in the neonatal mouse liver. (FIG. 2A) Number of protein-coding genes expressed in the liver as determined by RNA sequencing of livers at various time points. (FIG. 2B) Scheme for performing a genome-wide screen for hepatocyte fitness in neonatal mice. (FIG. 2C) Representation of sgRNAs in livers four days after injection with lentiviral library relative to the sgRNA representation in the plasmid library expressed as reads per million (RPM). n=2 male and 2 female mice. Pearson correlation r=0.97. (FIG. 2D) Pairwise comparisons of median fold change (log2) for each gene for each mouse at the endpoint of the screen. (FIG. 2E) Genes ranked by median fold change of gene scores across mice (log2) with significantly depleted genes denoted by red points and significantly enriched genes denoted by blue points (FDR<0.05 by two-sample Wilcoxon test). (FIG. 2F) Core essential genes (red bars) positioned based on gene rank as in (E) revealing their significant depletion in individual mice and across mice. p<2.2×10⁻¹⁶ for each mouse and across mice by one-sided Kolmogorov-Smirnov test. (FIG. 2G) Adjusted Wilcoxon p-value (−log10) versus the median fold change of gene scores across mice (log2) for each gene in the screen. Highlighted are control gene sets consisting of tumor suppressor genes in hepatocellular carcinoma (expected to enrich, blue) and genes required for hepatocyte viability (expected to deplete, red). Expected depleted p=3.5×10⁻⁶ by one-sided Kolmogorov-Smirnov test, expected enriched p=4.7×10⁻⁴ by one-sided Kolmogorov-Smirnov test.

FIGS. 3A-3D. An in vivo screen uncovers sex-dependent fitness effects and canonical tumor suppressor genes. (FIG. 3A) Median fold change (log2) across males versus median fold change (log2) across females for each gene. Highlighted are genes uniquely enriched in females (blue), genes uniquely enriched in males (cyan), genes uniquely depleted in females (red), and genes uniquely depleted in males (pink). Point size is proportional to the absolute difference in median log2 fold change between females and males. (FIG. 3B) Scheme for in vivo competition between hepatocytes expressing sgAAVS1-mTurq2 and sgCxorf38-mCherry (top panel). Ratio (log2) of sgCxorf38-mCherry-positive to sgAAVS1-mTurq2-positive hepatocytes in livers from male and female mice 0 or 3 weeks after Cas9 induction (bottom panel). n=one mouse per gender and >200 hepatocytes per mouse for 0 week time point and two mice per gender and >350 hepatocytes per mouse for 3 week time point. *p=0.016 (female 0 weeks versus female 3 weeks) by one-tailed Fisher's exact test. (FIG. 3C) Cumulative fraction of tumor suppressor genes (cyan, blue) and other genes (black, grey) based on quantile-normalized median fold change (log2) of their gene scores across screens in mouse embryonic stem (ES) cells and our screen. ES cells p>0.05 by one-sided Kolmogorov-Smirnov test, our screen p=6.5×10⁴ by one-sided Kolmogorov-Smirnov test. (FIG. 3D) Cumulative fraction of tumor suppressor genes (cyan, blue) and other genes (black, grey) based on quantile-normalized median fold change (log2) of their gene scores across screens in human hepatocellular carcinoma (HCC) cell lines and our screen. HCC cells p>0.05 by one-sided Kolmogorov-Smirnov test, our screen p=0.0028 by one-sided Kolmogorov-Smirnov test.

FIGS. 4A-4F. Class I MHC is essential for hepatocyte fitness. (FIG. 4A) KEGG gene sets exhibiting significant depletion (FDR q-value<0.05) at the endpoint of the screen ranked by FDR q-value (−log10). Bars extending to the end of the plot indicate an FDR q-value of 0. (FIG. 4B) KEGG gene sets exhibiting significant depletion (FDR q-value<0.05) in our screen relative to screens in either mouse embryonic stem (ES) cells (dark grey bars) or human hepatocellular carcinoma (HCC) cell lines (light grey bars) ranked by FDR q-value (−log10). Bars extending to the end of the plot indicate an FDR q-value of 0. (FIG. 4C) Quantile-normalized median fold change (log2) for genes in the KEGG gene set for glycosaminoglycan biosynthesis and heparan sulfate in ES cell screens, HCC cell line screens, and our screen. Genes uniquely depleted in our screen are highlighted in red. The bounds of the box indicate the first and third quartiles, and the whiskers extend to the furthest data point that is within 1.5 times the interquartile range. (FIG. 4D) Quantile-normalized median fold change (log2) for genes in the KEGG gene set for antigen processing and presentation in ES cell screens, HCC cell line screens, and our screen. Genes uniquely depleted in our screen are highlighted in red. The bounds of the box indicate the first and third quartiles, and the whiskers extend to the furthest data point that is within 1.5 times the interquartile range. (FIG. 4E) Pathway for presentation of peptide antigens on class I MHC complexes. Created with BioRender.com. (FIG. 4F) Scheme for in vivo competition between hepatocytes expressing sgAAVS1-mTurq2 and sgTap1-mCherry (top panel). Ratio (log2) of sgTap1-mCherry-positive to sgAAVS1-mTurq2-positive hepatocytes in livers from mice at 0 or 3 weeks after Cas9 induction without or with NK cell depletion (bottom panel). Closed and open circles represent values from male and female mice, respectively. n=one male and one female mouse per time point and >100 hepatocytes per mouse for 0 week time point and >200 hepatocytes per mouse for 3 week time point. *p=0.026 (3 weeks without NK depletion versus 3 weeks with NK depletion) by one-tailed Fisher's exact test.

FIGS. 5A-5K. Efficient delivery of sgRNAs and depletion of long-lived proteins in the liver. (FIG. 5A) Table of values used to estimate number of hepatocytes in postnatal day one livers. Liver volume was measured by volume displacement and percent hepatocytes and hepatocyte volume were measured by immunostaining and microscopy. (FIG. 5B) Images of endogenous mCherry and mTurq2 fluorescence in livers from mice four days or 14 weeks after injection of 2.5×10⁷ TU of an equal mixture of sgAAVS1-mCherry and sgAAVS1-mTurq2 lentiviruses. Scale bars, 100 μm. (FIG. 5C) Percent mCherry-, mTurq2-, and double-positive hepatocytes in livers from mice four days or 14 weeks after injection of 2.5×10⁷ TU of an equal mixture of sgAAVS1-mCherry and sgAAVS1-mTurq2 lentiviruses. n=3 mice per time point and 200 hepatocytes per mouse. Error bars indicate standard deviation. (FIG. 5D) Images of livers from LSL-Cas9 mice injected with 0 or 2×10¹¹ GC of AAV-Cre on postnatal day five and harvested four days thereafter immunostained for ASGR1 (hepatocyte marker, magenta) and Cas9 (green) and counterstained with Hoechst (blue). Scale bars, 15 μm. (FIG. 5E) Percent Cas9-positive hepatocytes in livers from LSL-Cas9 mice injected with varying doses of AAV-Cre on postnatal day five and harvested four days thereafter as determined by Cas9 immunostaining. n=1 male and 1 female mouse per dose and 200 hepatocytes per mouse. Error bars indicate standard deviation. (FIG. 5F) Images of livers from LSL-Cas9 mice injected with sgLmnb2-mCherry followed by PBS or AAV-Cre immunostained for mCherry (magenta) and lamin B2 (green) and counterstained with Hoechst (blue). Scale bars, 45 μm. (FIG. 5G) Nuclear lamin B2 intensity per μm in mCherry-positive and mCherry-negative hepatocytes from LSL-Cas9 mice injected with sgLmnb2-mCherry followed by PBS or AAV-Cre. n=1 male and 1 female mouse per condition and 25 cells per mouse. Closed and open circles represent values from male and female mouse, respectively. (FIG. 5H) Images of livers from unperturbed postnatal day 12 LSL-Cas9 mice or postnatal day 12 LSL-Cas9 mice injected with 1.25×10⁷ TU of sgMaob-mCherry or sgLmnb2-mCherry lentivirus on postnatal day 1 followed by AAV-Cre on postnatal day 5 immunostained for CD45 (green) and mCherry (magenta) and counterstained for Hoechst (blue). Scale bars, 45 μm. (FIG. 5I) Number of CD45-positive cells per 40× field in unperturbed postnatal day 12 LSL-Cas9 mice or postnatal day 12 LSL-Cas9 mice injected with 1.25×10⁷ TU of sgMaob-mCherry or sgLmnb2-mCherry lentivirus on postnatal day 1 followed by AAV-Cre on postnatal day 5 as determined by CD45 immunostaining n =2 male and 2 female mice per condition and five fields per mouse. Closed and open circles represent values from male and female mice, respectively. (FIG. 5J) Images of livers from unperturbed postnatal day 12 LSL-Cas9 mice or postnatal day 12 LSL-Cas9 mice injected with 1.25×10⁷ TU of sgMaob-mCherry or sgLmnb2-mCherry lentivirus on postnatal day 1 followed by AAV-Cre on postnatal day 5 immunostained for Ki67 (white), mCherry (magenta) and actin (green) and counterstained for Hoechst (blue). Scale bars, 45 μm. (FIG. 5K) Percent Ki67-positive hepatocytes in livers from unperturbed postnatal day 12 LSL-Cas9 mice or postnatal day 12 LSL-Cas9 mice injected with 1.25×10⁷ TU of sgMaob-mCherry or sgLmnb2-mCherry lentivirus on postnatal day 1 followed by AAV-Cre on postnatal day 5 as determined by Ki67 immunostaining. n=2 male and 2 female mice per condition and 50 cells per mouse. Closed and open circles represent values from male and female mice, respectively.

FIGS. 6A-6F. A genome-wide screen for hepatocyte fitness in the neonatal mouse liver. (FIG. 6A) Time points during liver growth, quiescence, and regeneration from which livers were harvested for RNA sequencing. Partial hepatectomy and carbon tetrachloride were used as surgical resection and toxic injury models, respectively. (FIG. 6B) Average RNA expression (FPKM) of representative protein-coding genes at different time points of liver growth, quiescence, and regeneration. Dashed line indicates FPKM cutoff of 0.3. n=3 male mice per time point (FIG. 6C) Number of sgRNAs with a given representation (log10 RPM) for all sgRNAs in the library. (FIG. 6D) Pearson correlation (r) for each plot in FIG. 2D. (FIG. 6E) Average RNA expression at postnatal day 15 (log10 FPKM) relative to median fold change across mice (log2) for each gene. Spearman p=−0.15. (FIG. 6F) Average protein half-life (log10 hours) relative to median fold change across mice (log2) for each gene. Spearman p=0.01.

FIG. 7. An in vivo screen uncovers sex-dependent fitness effects and canonical tumor suppressor genes. Western blot showing Cxorf38 protein levels in Cas9-AML12 cells stably transduced with sgAAVS1, sgTap1, or sgCxorf38. A nonspecific band is shown as a loading control.

FIGS. 8A-8D. Class I MHC is essential for hepatocyte fitness. (FIG. 8A) Quantile-normalized median fold change (log2) of genes in the KEGG gene set for protein export in ES cell screens, HCC cell line screens, and our screen. Genes depleted in more than one screen are highlighted in blue. The bounds of the box indicate the first and third quartiles, and the whiskers extend to the furthest data point that is within 1.5 times the interquartile range. (FIG. 8B) Quantile-normalized median fold change (log2) for genes in the KEGG gene set for SNARE interactions in vesicular transport in ES cell screens, HCC cell line screens, and our screen. Genes depleted in more than one screen are highlighted in blue. The bounds of the box indicate the first and third quartiles, and the whiskers extend to the furthest data point that is within 1.5 times the interquartile range. (FIG. 8C) Median fold change (log2) across mice for genes associated with class I MHC or class II MHC within the KEGG gene set for antigen processing and presentation in our screen. The bounds of the box indicate the first and third quartiles, and the whiskers extend to the furthest data point that is within 1.5 times the interquartile range. (FIG. 8D) Western blot showing Tap1 protein levels in Cas9-AML12 cells stably transduced with sgAAVS1 or sgTap1. A nonspecific band is shown as a loading control.

### DETAILED DESCRIPTION OF THE INVENTION

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Methods of Generating an In Vivo Library

Some aspects of the present disclosure are directed to a method for generating an in vivo library for genomic screening, comprising providing a non-human mammal comprising cells in an organ or tissue having a sequence (e.g., having a sequence integrated into the genome) encoding a Cas protein or a functional portion thereof, introducing a plurality of single guide RNAs (sgRNA) into the non-human mammal with a viral vector to obtain an in vivo library for genomic screening comprising cells of the tissue or organ, wherein each cell comprises a sequence encoding a single guide RNA of the plurality of single guide RNAs.

The non-human mammal is not limited. In some embodiments, the non-human mammal is selected from non-human primates (gorilla, chimpanzee, orangutan, macaque, gibbon), domestic animals (dog and cat), farm and ranch animals (horse, cow, goat, sheep, pig), rodents, and laboratory and experimental animals (mouse, rat, rabbit, guinea pig). In some embodiments, the non-human mammal is a mouse or rat. In some embodiments, the non-human animal is a mouse. In some embodiments, the non-human animal is an adult, neonate, or embryo. In some embodiments, the non-human mammal is neonate or embryo. In some embodiments, the neonate is a non-human mammal (e.g., mouse) at postnatal day (PD) 1, 2, 3, 4, 5, 6, 7, 8, 9, or 10. In some embodiments, the neonate is a mouse at PD1. In some embodiments, the neonate is a mouse at PD2.

In some embodiments, the non-human mammal has a disease or condition, is predisposed to a disease or condition, or has a genetic abnormality The disease or condition is not limited. In some embodiments the disease or condition is a proliferative disease or condition, an inflammatory disease or condition, a cardiovascular disease or condition, a neurological disease or condition, a metabolic disease or condition (e.g., metabolic disease) or an infectious disease. The genetic abnormality is also not limited. In some embodiments, the genetic abnormality is associated with a disease or condition (e.g., a disease or condition provided herein).

The organ or tissue is not limited. In some embodiments, the organ is a solid organ. In some embodiments, the organ or tissue is adipose, brain, epithelial, colon, heart, kidney, liver, lung, muscle, nerve, ovary, pancreas, small intestine, large intestine, spleen, stomach, testis, or uterus. In some embodiments, the organ or tissue is the liver or hepatic tissue. In some embodiments, the cells are not proliferating. In some embodiments, the cells are proliferating.

The Cas protein or functional fragment thereof is not limited and may be any suitable Cas protein or functional fragment having a desired activity. Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell to allow for recognition and modification of sites comprising the same, similar or different PAM motifs.

In some embodiments, the Cas protein is Cpf1 protein or a functional portion thereof. In some embodiments, the Cas protein is Cpf1 from any bacterial species or functional portion thereof. In certain embodiments, a Cpf1 protein is a Francisella novicida U112 protein or a functional portion thereof, an Acidaminococcus sp. BV3L6 protein or a functional portion thereof, or a Lachnospiraceae bacterium ND2006 protein or a function portion thereof. Cpf1 protein is a member of the type V CRISPR systems. Cpf1 protein is a polypeptide comprising about 1300 amino acids. Cpf1 contains a RuvC-like endonuclease domain In some embodiments, the Cas protein is inactive. In some embodiments, the catalytically inactive Cas protein is a Cas9 protein Amino acids mutations that create a catalytically inactive Cas9 protein include mutating at residue 10 and/or residue 840. Mutations at both residue 10 and residue 840 can create a catalytically inactive Cas9 protein, sometimes referred to herein as dCas9. For example, a D10A and a H840A Cas9 mutant is catalytically inactive.

In some embodiments, the Cas protein or functional fragment thereof is selected from the group consisting of Cas9, or a catalytically inactive Cas protein fused to an effector domain.

As used herein an “effector domain” is a molecule (e.g., protein) that modulates the expression and/or activation of a genomic sequence (e.g., gene). The effector domain may have methylation activity or demethylation activity (e.g., DNA methylation or DNA demethylation activity). In some aspects, the effector domain targets one or both alleles of a gene. The effector domain can be introduced as a nucleic acid sequence and/or as a protein. In some aspects, the effector domain can be a constitutive or an inducible effector domain In some aspects, a Cas (e.g., dCas) nucleic acid sequence or variant thereof and an effector domain nucleic acid sequence are introduced into a cell. In some aspects, the effector domain is fused to a molecule that associates with (e.g., binds to) Cas protein (e.g., the effector molecule is fused to an antibody or antigen binding fragment thereof that binds to Cas protein). In some aspects, a Cas (e.g., dCas) protein or variant thereof and an effector domain are fused or tethered creating a chimeric protein and are introduced into the cell as the chimeric protein. In some aspects, the Cas (e.g., dCas) protein and effector domain bind as a protein-protein interaction. In some aspects, the Cas (e.g., dCas) protein and effector domain are covalently linked. In some aspects, the effector domain associates non-covalently with the Cas (e.g., dCas) protein. In some aspects, a Cas (e.g., dCas) nucleic acid sequence and an effector domain nucleic acid sequence are introduced as separate sequences and/or proteins. In some aspects, the Cas (e.g., dCas) protein and effector domain are not fused or tethered.

The effector domain is not limited and may be any suitable effector domain In some embodiments, the effector domain is selected from the group consisting of a transcription activation domain, transcriptional repression domain, chromatin organizer domain, a remodeler domain, a histone modifier domain, a DNA modification domain, an RNA binding domain, a protein interaction input device domain, and a protein interaction output device domain.

Examples of effector domains include a transcription activation domain (e.g, Ga14, Oaf1, Leu3, Rtg3, Pho4, Gln3, Gcn4, p53, NFAT, NF-ÿB, or VP16 transcription activation domain), chromatin organizer domain, a remodeler domain, a histone modifier domain, a DNA modification domain, a RNA binding domain, a protein interaction input device domain (Grunberg and Serrano, Nucleic Acids Research, 3 '8 (8): '2663-267 '5 (2010)), and a protein interaction output device domain (Grunberg and Serrano, Nucleic Acids Research, 3 '8 (8): '2663-267 '5 (2010)). In some aspects, the effector domain is a DNA modifier. Specific examples of DNA modifiers include 5hmc conversion from 5mC such as Tet1 (Tet1CD); DNA demethylation by Tet1, ACID A, MBD4, Apobec1, Apobec2, Apobec3, Tdg, Gadd45a, Gadd45b, ROS1; DNA methylation by Dnmt1, Dnmt3a, Dnmt3b, CpG Methyltransferase M.SssI, and/or M.EcoHK31I. In specific aspects, an effector domain is Tet1. In other specific aspects, as effector domain is Dmnt3a. In some embodiments, dCas9 is fused to Tet1. In other embodiments, dCas9 is fused to Dnmt3a. Other examples of effector domains are described in PCT Application No. PCT/US2014/034387 and U.S. application Ser. No. 14/785,031, which are incorporated herein by reference in their entirety. Methods of using catalytically inactive Cas proteins and effector domains for modifying a nucleotide sequence (e.g., genomic sequence), and sgRNA are taught in PCT/US2017/065918 filed 12 Dec. 2017, which is incorporated herein by reference.

In some embodiments, the Cas protein or functional fragment thereof comprises a detectable label. The term “detectable tag” or “detectable label” as used herein includes, but is not limited to, detectable labels, such as fluorophores, radioisotopes, colorimetric substrates, or enzymes; heterologous epitopes for which specific antibodies are commercially available, e.g., FLAG-tag; heterologous amino acid sequences that are ligands for commercially available binding proteins, e.g., Strep-tag, biotin; fluorescence quenchers typically used in conjunction with a fluorescent tag on the other polypeptide; and complementary bioluminescent or fluorescent polypeptide fragments. A tag that is a detectable label or a complementary bioluminescent or fluorescent polypeptide fragment may be measured directly (e.g., by measuring fluorescence or radioactivity of, or incubating with an appropriate substrate or enzyme to produce a spectrophotometrically detectable color change for the associated polypeptides as compared to the unassociated polypeptides). A tag that is a heterologous epitope or ligand is typically detected with a second component that binds thereto, e.g., an antibody or binding protein, wherein the second component is associated with a detectable label. In some embodiments, the detectable tag is a fluorescent tag.

In some embodiments, expression of the Cas protein or functional fragment thereof is under control of an inducible promoter and/or wherein expression of the plurality of sgRNAs are under control of an inducible promoter. The term “inducible promoter”, as used herein, refers to a promoter that, in the absence of an inducer (such as a chemical and/or biological agent), does not direct expression, or directs low levels of expression of an operably linked gene (including cDNA), and, in response to an inducer, its ability to direct expression is enhanced. Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotic (e.g., tetracycline or doxycycline).

In some specific embodiments, expression of the Cas protein or functional fragment thereof is induced with a site-specific recombinase. In some specific embodiments, expression of the plurality of sgRNAs are induced with a site-specific recombinase. The term “site-specific recombinase” (also referred to simply as a “recombinase” herein) refers to a protein that can recognize and catalyze the recombination of DNA between specific sequences in a DNA molecule. Such sequences may be referred to as “recombination sequences” or “recombination sites” for that particular recombinase. Tyrosine recombinases and serine recombinases are the two main families of site-specific recombinase. Examples of site-specific recombinase systems include the Cre/Lox system (Cre recombinase mediates recombination between loxP), the Flp/Frt system (Flp recombinase mediates recombination between FRT sites), and the PhiC31 system (PhiC31 recombinase mediates DNA recombination at sequences known as attB and attP sites). Recombinase systems similar to Cre include the Dre-rox, VCre/VloxP, and SCre/SloxP systems (Anastassiadis K, et al. (2009) Dis Model Mech 2(9-10):508-515; Suzuki E, Nakayama M (2011) Nucl. Acids Res. (2011) 39 (8): e49. It should be understood that reference to a particular recombinase system is intended to encompass the various engineered and mutant forms of the recombinases and recombination sites and codon-optimized forms of the coding sequences known in the art. DNA placed between two loxP sites is said to be “foxed”. A gene may be modified by the insertion of two loxP sites that allow the excision of the floxed gene segment through Cre-mediated recombination. In some embodiments, expression of Cre may be under control of a cell type specific, cell state specific, or inducible expression control element (e.g., cell type specific, cell state specific, or inducible promoter) or Cre activity may be regulated by a small molecule. For example, Cre may be fused to a ligand binding domain of a receptor (e.g., a steroid hormone receptor) so that its activity is regulated by receptor ligands. Cre-ER(T) or Cre-ER(T2) recombinases may be used, which comprise a fusion protein between a mutated ligand binding domain of the human estrogen receptor (ER) and Cre, the activity of which can be induced by, e.g., 4-hydroxy-tamoxifen. Placing Lox sequences appropriately allows a variety of genomic manipulations. In some embodiments, a nucleotide sequence coding for the site-specific recombinase (e.g., Cre) is introduced into the cell. In some embodiments, a nucleotide sequence coding for the site-specific recombinase (e.g., Cre) is introduced with a viral vector (e.g., AAV vector).

sgRNAs are known in the art. sgRNA refers to a single, contiguous RNA sequence that interacts with a cognate Cas protein equivalently as described for tracrRNA/crRNA polynucleotides. For example, a Cas9 single-guide RNA (Cas9-sgRNA) is a guide RNA wherein the Cas9-crRNA is covalently joined to the Cas9-tracrRNA, often through a tetraloop, and forms an RNA polynucleotide secondary structure through base-pair hydrogen bonding. See, e.g., Jinek, et al, Science (2012) 337:816-821; PCT Publication No. WO 2013/176772, published Nov. 28, 2013; (each of which is incorporated herein by reference in its entirety).

In some embodiments, the plurality of sgRNA target each of the expressed genes of one or more pathways of the cells. The pathways are not limited. In some embodiments, the cellular pathway is a pathway regulating tissue or organ regeneration, cell metabolism, cell activation, cell proliferation, cell differentiation, cell mutation, cell cycle and cell death. In some embodiments, the plurality of sgRNA target the expressed genes of the cells or a substantial portion thereof. In some embodiments, the plurality of sgRNA target at least 80%, 85%, 90%, 95%, 97%, 99%, 99.9%, 99.99%, 99.995%, or 99.999% of all the genes of a cell. In some embodiments, the plurality of sgRNA target at least 80%, 85%, 90%, 95%, 97%, 99%, 99.9%, 99.99%, 99.995%, or 99.999% of all the expressed genes of a cell.

In some embodiments, the plurality of sgRNA target at least 80%, 85%, 90%, 95%, 97%, 99%, 99.9%, 99.99%, 99.995%, or 99.999% of all the not expressed genes of a cell. In some embodiments, the plurality of sgRNA target at least, 20, 50, 100, 500, 1000, 5000, 8000, 10,000 or more genes (e.g., expressed genes, not expressed genes).

In some embodiments, it is desirable to have 2 or more sgRNAs targeting the same expressed gene or other genomic site in order to confirm that a phenotypic change associated with an sgRNA is due to modification of the expressed gene or genomic site and not due to other factors such as off-target effects. In some embodiments, an average of more than 1.5 sgRNA species target each expressed gene or genomic site. In some embodiments, an average of more than two sgRNA species target each expressed gene or genomic site. In some embodiments, an average of more than three sgRNA species target each expressed gene or genomic site. In some embodiments, an average of more than four sgRNA species target each expressed gene or genomic site. In some embodiments, an average of five or more sgRNA species target each expressed gene or genomic site.

In some embodiments, it is desirable to use an sgRNA having a known effect with the Cas protein or functional fragment to use as a control. For example, a gRNA causing the cleavage of an expressed gene required for cell survival may be used to ensure assay fidelity. In some embodiments, the plurality of sgRNA comprise one or more species (e.g., 1, 2, 3, 4, 5, or more) targeting one or more control genes.

The viral vector may be introduced (i.e., administered) by any suitable means. In some embodiments, the viral vector is administered orally, parenterally (intramuscularly, intravenously, subcutaneously), or locally to a tissue or organ. In some embodiments, the viral vector is introduced by injection into a vein. In some embodiments, the vein is a superficial vein. In some embodiments, the vein is a temporal vein. In some embodiments, the viral vector is introduced by injection into the temporal vein of an infant non-human animal (e.g., at PD1 of a mouse).

The viral vector is not limited and may be any suitable viral vector. In some embodiments, the viral vector integrates a nucleotide sequence encoding the sgRNA into the genome of the cells. Any suitable viral vector with genomic integration activity may be used. In some embodiments, the viral vector is a lentiviral vector or retroviral vector.

The appropriate dose of viral vector administered will depend on tissue or organ, viral vector, and intended usage and can be routinely determined in view of the teachings of the present disclosure. In some embodiments, about 1-10*10⁷ transduction units of viral vector are introduced into the non-human mammal In some embodiments, about 4-6*10⁷ transduction units of viral vector are introduced into the non-human mammal

In some embodiments, about 5*10⁷ transduction units of viral vector are introduced into the non-human mammal (e.g., PD1 mouse). In some embodiments, after administration of the viral vector about 10%, 20%, 25%, 50%, 75%, or 90% of the cells in an organ or tissue comprise sgRNA (e.g., comprise sgRNA integrated into the genome). In some embodiments, about 75%, or more of the cells in an organ or tissue comprise sgRNA (e.g., comprise sgRNA integrated into the genome). In some embodiments, at least about 1 million, 2 million, 3 million, 4 million, 5 million, 6 million, 7 million, 8 million, 9 million, or 10 million cells in the organ or tissue (e.g., liver) comprise sgRNA (e.g., comprise sgRNA integrated into the genome).

Non-Human Animals

Some aspects of the present disclosure are directed to a non-human mammal comprising an in vivo library described herein or generated by the methods disclosed herein.

Some aspects of the present disclosure are directed to a non-human mammal comprising an in vivo library of a population of cells in an organ or tissue having a sequence encoding a Cas protein or a functional portion thereof and a sequence encoding an sgRNA of a plurality of sgRNAs targeting the genomic sites. In some embodiments, the non-human animal comprising an in vivo library is produced by a method described herein.

In some embodiments, the non-human mammal is a mouse or rat. In some embodiments, the non-human mammal is neonate or embryo. In some embodiments, the non-human mammal has a disease or condition, is predisposed to a disease or condition, or has a genetic abnormality In some embodiments, the organ or tissue is the liver or hepatic tissue. In some embodiments, the cells are proliferating.

In some embodiments, the Cas protein or functional fragment thereof is selected from the group consisting of Cas9, or a catalytically inactive Cas protein fused to an effector domain In some embodiments, the effector domain is selected from the group consisting of a transcription activation domain, transcriptional repression domain, chromatin organizer domain, a remodeler domain, a histone modifier domain, a DNA modification domain, an RNA binding domain, a protein interaction input devices domain, and a protein interaction output device domain In some embodiments, the Cas protein or functional fragment thereof comprises a detectable label. In some embodiments, expression of the Cas protein or functional fragment thereof is under control of an inducible promoter and/or wherein expression of the plurality of sgRNAs are under control of an inducible promoter.

In some embodiments, the plurality of sgRNA target each of the expressed genes of one or more pathways of the cells. In some embodiments, the plurality of sgRNA target the expressed genes of the cells or a substantial portion thereof. In some embodiments, an average of more than two sgRNA species target each expressed gene. In some embodiments, an average of five or more sgRNA species target each expressed gene. In some embodiments, the plurality of sgRNA target at least, 20, 50, 100, 500, 1000, 5000, or more expressed genes. In some embodiments, the plurality of sgRNA comprise one or more species targeting one or more control genes.

In some embodiments, about 10%, 20%, 25%, 50%, 75%, or 90% of the cells in an organ or tissue comprise sgRNA (e.g., comprise sgRNA integrated into the genome). In some embodiments, about 75%, or more of the cells in an organ or tissue comprise sgRNA (e.g., comprise sgRNA integrated into the genome). In some embodiments, at least about 1 million, 2 million, 3 million, 4 million, 5 million, 6 million, 7 million, 8 million, 9 million, or 10 million cells in the organ or tissue (e.g., liver) comprise sgRNA (e.g., comprise sgRNA integrated into the genome).

Methods of Screening

Some aspects of the present disclosure are directed to a method of in vivo screening for genomic sites in cells in an organ or tissue associated with a change in phenotype, comprising providing a non-human animal comprising an in vivo library of a population of cells in an organ or tissue having a sequence encoding a Cas protein or a functional portion thereof and a sequence encoding an sgRNA of a plurality of sgRNAs targeting the genomic sites, providing conditions under which the Cas protein or functional portion thereof and the sgRNA contact and modify the genomic sites, detecting one or more phenotypic changes in the population of cells and determining a genomic site associated with the phenotypic change by identifying a sgRNA associated with the phenotypic change and determining the sgRNA target genomic site.

The genomic sites targeted by the sgRNAs are not limited. In some embodiments, the genomic sites are expressed genes, gene regulatory elements, methylation sites, or gene silencing sites. In some embodiments, the genomic sites are expressed genes in the population of cells. In some embodiments, the sgRNA target each of the expressed genes of one or more pathways of the cells. In some embodiments, the sgRNA target the expressed genes of the cells or a substantial portion thereof. In some embodiments, an average of more than two sgRNA species target each expressed gene. In some embodiments, an average of five or more sgRNA species target each expressed gene. In some embodiments, the plurality of sgRNA target at least, 20, 50, 100, 500, 1000, 5000, or more expressed genes. In some embodiments, the comprise one or more species targeting one or more control genes.

The phenotypic change is not limited and may be any suitable phenotypic change. In some embodiments, the phenotype is survival, proliferation, resistance or susceptibility to an agent (e.g., a toxin, drug, biologic, or chemotherapeutic agent), cell differentiation, morphology, expression of a gene or protein, protein localization, or resistance or susceptibility to a disease or condition (e.g., any disease or condition described herein). In some embodiments, in methods that suppress expression of an sgRNA target, the phenotypic change can be a reduction in survival or proliferation caused by suppression of expression of a gene (or genes), or an increase in survival or proliferation caused by suppression of expression of a gene (or genes). In some embodiments, in methods that suppress expression of an sgRNA target and wherein the non-human animal is exposed to a toxin or biologic agent (e.g., infectious agent), the phenotypic change can be increased or decreased susceptibility to the toxin or infectious agent.

The non-human mammal is not limited and may be any non-human mammal described herein. In some embodiments, the non-human mammal is a mouse or rat. In some embodiments, the non-human mammal is neonate or embryo (e.g., a PD1 mouse). In some embodiments, the non-human mammal has a disease or condition, is predisposed to a disease or condition, has a genetic abnormality, or has been contacted with an agent. The disease or condition is not limited and may be any disease or condition described herein. The genetic abnormality is not limited and may be any genetic abnormality described herein. In some embodiments, the agent is a toxin, drug, biologic agent, or chemotherapeutic agent. The organ or tissue is not limited and may be any organ or tissue described herein. In some embodiments, the organ or tissue is the liver or hepatic tissue. In some embodiments, the population of cells is non-proliferative. In some embodiments, the population of cells is proliferating.

The Cas protein or functional fragment thereof is not limited and may be any Cas protein or functional fragment thereof described herein. In some embodiments, the Cas protein or functional fragment thereof is selected from the group consisting of Cas9, or a catalytically inactive Cas protein fused to an effector domain In some embodiments, wherein the effector domain is selected from the group consisting of a transcription activation domain, transcriptional repression domain, chromatin organizer domain, a remodeler domain, a histone modifier domain, a DNA modification domain, a RNA binding domain, a protein interaction input devices domain, and a protein interaction output device domain In some embodiments, the Cas protein or functional fragment thereof comprises a detectable label. In some embodiments, expression of the Cas protein or functional fragment thereof is under control of an inducible promoter.

In some embodiments, expression of the plurality of sgRNAs are under control of an inducible promoter. In some embodiments, the plurality of sgRNA target each of the expressed genes of one or more pathways of the population of cells. In some embodiments, the plurality of sgRNA target the expressed genes of the population of cells or a substantial portion thereof. In some embodiments, an average of more than two sgRNA species target each expressed gene. In some embodiments, an average of five or more sgRNA species target each expressed gene. In some embodiments, the plurality of sgRNA target at least, 20, 50, 100, 500, 1000, 5000, or more expressed genes. In some embodiments, the plurality of sgRNA comprise one or more species targeting one or more control genes. In some embodiments, about 10%, 20%, 25%, 50%, 75%, or 90% of cells in the organ or tissue comprise sgRNA.

In some embodiments, the conditions comprise inducing expression of the Cas protein or functional portion thereof and/or the plurality of sgRNAs. The methods of inducing expression are not limited and may be any method described herein. In some embodiments, expression is induced with an inducible promoter. In some embodiments, expression is induced with a site-specific recombinase (e.g., Cre/lox). In some embodiments, expression is induced by administration of a viral vector (e.g., AAV, AAV8) comprising a nucleic acid coding for a site-specific recombinase (e.g., Cre).

In some embodiments, the population of cells constitutively express the plurality of sgRNAs and wherein the conditions comprise inducing expression of the Cas protein or functional portion thereof. In some embodiments, the expression of the Cas protein or functional portion thereof is under control of a Cre-lox system.

In some embodiments, the period of time between the performance of providing conditions under which the Cas protein or functional portion thereof and the sgRNA contact and modify the genomic sites and detecting one or more phenotypic changes in the population of cells is sufficient to allow for one or more divisions of the cells of the population of cells. The period of time sufficient to allow for one or more divisions of the cells will vary depending on the non-human mammal and the tissue or organ. In some embodiments, the period of time is sufficient to allow for at least two divisions of the cells of the population of cells. In some embodiments, the period of time is sufficient to allow for at least three divisions of the cells of the population of cells. In some embodiments, the period of time is sufficient to allow for at least four divisions of the cells of the population of cells. In some embodiments, the period of time is sufficient to allow for at least five divisions of the cells of the population of cells. In some embodiments, the period of time is not sufficient to allow for one divisions of the cells of the population of cells (e.g., the detection of phenotypic changes occurs immediately after the providing of conditions under which the Cas protein or functional portion thereof and the sgRNA contact and modify the genomic sites). In some embodiments, the period of time is about 1 hour, 6 hours, 12 hours, 18 hours, 1 day, 2 days, 3 days, 1 week, 2 weeks, 1 month, 2 months, 6 months, 1 year, 2 years, or longer.

In some embodiments, the step of detecting one or more phenotypic changes in the population of cells comprises harvesting the population of cells and detecting the relative abundance of each sgRNA species in the population of cells. In some embodiments, enrichment and/or depletion (e.g., as compared to the remainder of the gRNA in the population), or degrees of enrichment and/or depletion, are identified. Methods of detecting the relative abundance of each sgRNA are not limited and may be any suitable method. In some embodiments, the relative abundance of each gRNA is detected by sequencing, e.g., next generation sequencing (NGS). In some embodiments, the sequencing method is multiplexed PCR-based NGS. In some embodiments, the sequencing method is a comprehensive amplicon NGS.

Next-generation sequencing technologies can include any one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing and/or other suitable semiconductor-based sequencing technologies, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), sequencing-by-synthesis, tunneling currents sequencing, sequencing by hybridization, mass spectrometry sequencing, microscopy-based techniques, and/or any suitable next-generation sequencing technologies.

Additionally or alternatively, sequencing technologies can include any one or more of: capillary sequencing, Sanger sequencing (e.g., microfluidic Sanger sequencing, etc.), pyrosequencing, nanopore sequencing (Oxford nanopore sequencing, etc.), and/or any other suitable types of sequencing facilitated by any suitable sequencing technologies.

In some embodiments, the method further includes identifying, based on the relative abundance for each sgRNA, enrichment and/or depletion of genomic regions (e.g., expressed genes) in the population of cells that are the targets for the enriched or depleted sgRNA. In some embodiments, the identified genomic regions are further assessed, e.g., in drug discovery screens, animal models of diseases, etc.

For example, in some embodiments, the method can be used to identify a genomic region (e.g., expressed gene) that confers reduced susceptibility to a toxin or infectious agent as, in some embodiments, sgRNAs that target the genomic region would be depleted in a population of cells that is also contacted with the toxin or infectious agent. Thus, screens that identify compounds which enhance the level or activity of a gene product of the identified region and thus suppress the activity of the toxin or infectious agent could be performed.

Alternatively, in some embodiments, the method can be used to identify a genomic region (e.g., expressed gene) that confers increased susceptibility to a toxin or infectious agent as, in some embodiments, sgRNAs that target the genomic region would be enriched in a population of cells that is also contacted with the toxin or infectious agent. Thus, screens that identify compounds which reduce the level or activity of a gene product of the identified region and thus suppress the activity of the toxin or infectious agent could be performed. For example, in some embodiments, the method may identify an expressed gene coding for a receptor that is necessary for entry of the toxin or infectious agent into the cell. In such case, sgRNAs targeting a gene expressing the receptor may be enriched in a population of cells contacted with the toxin or infectious agent.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

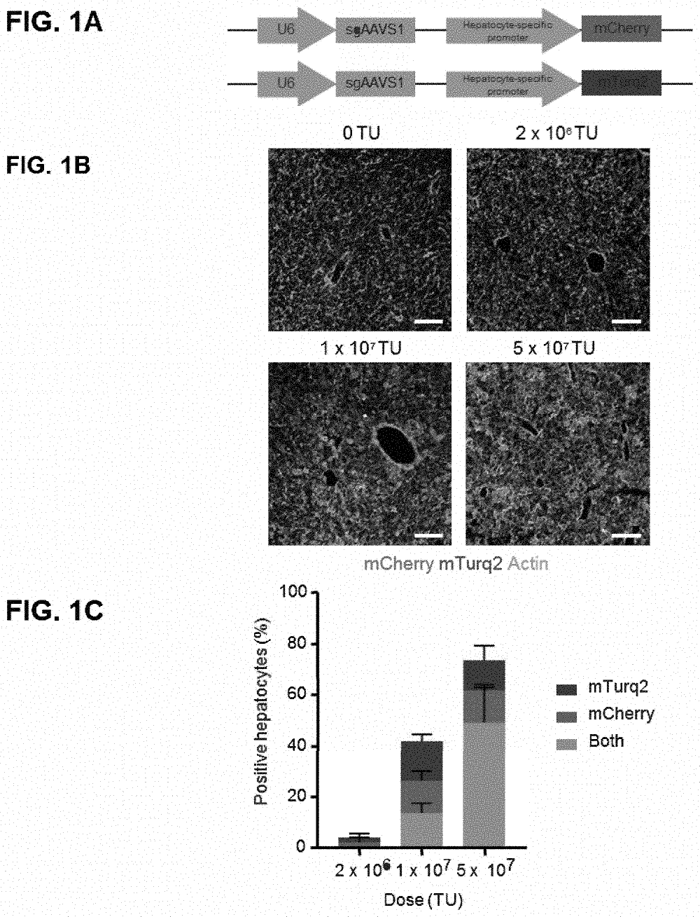

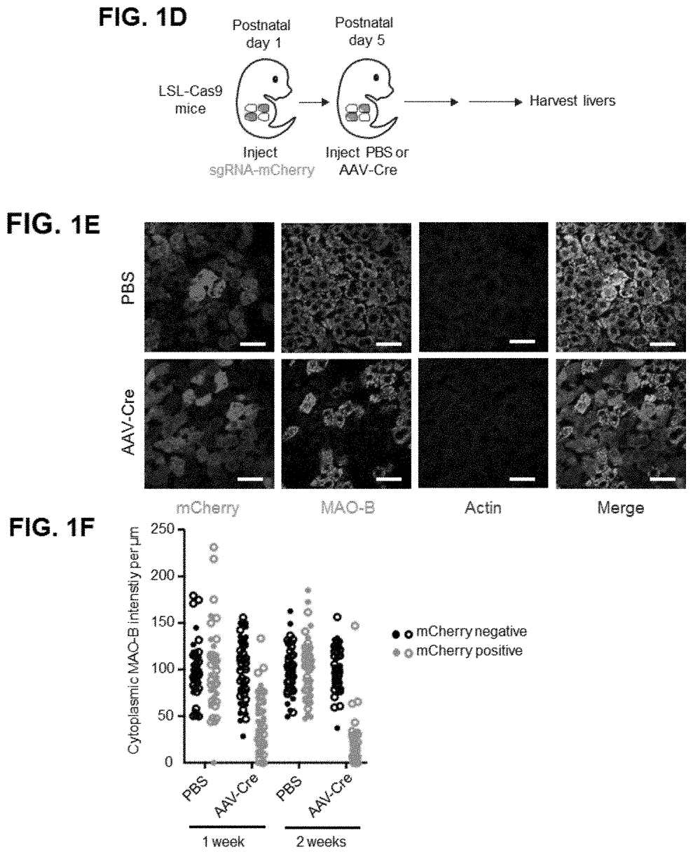

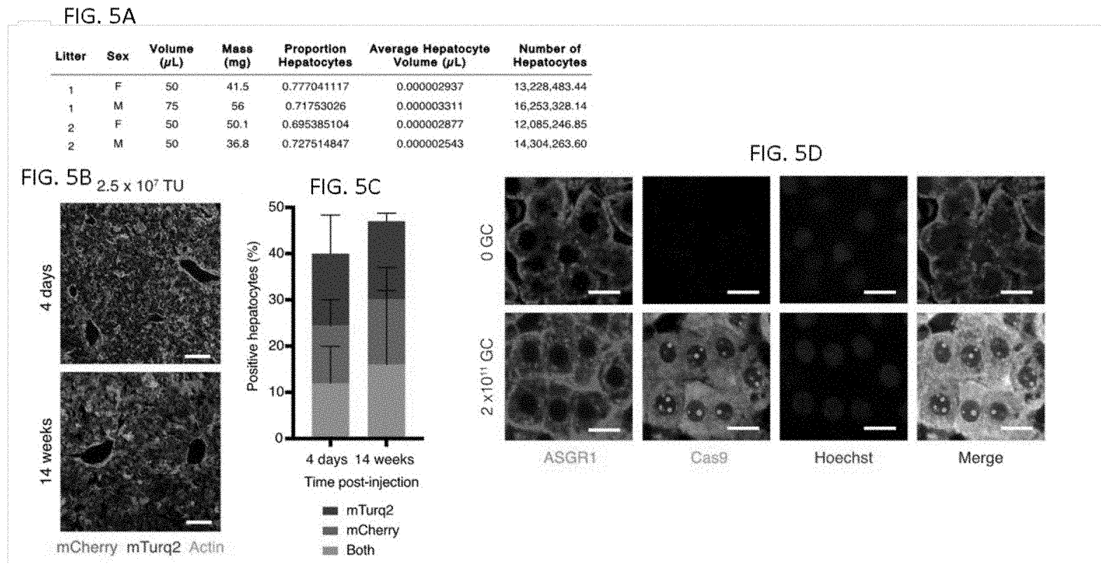

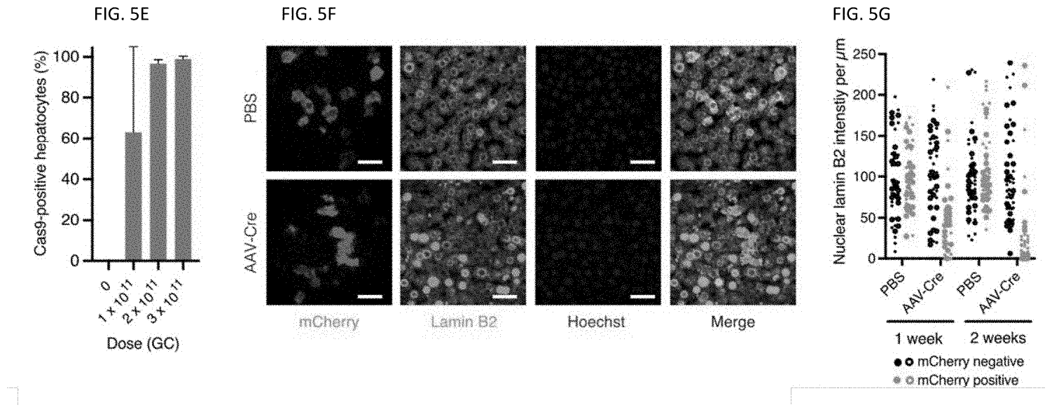

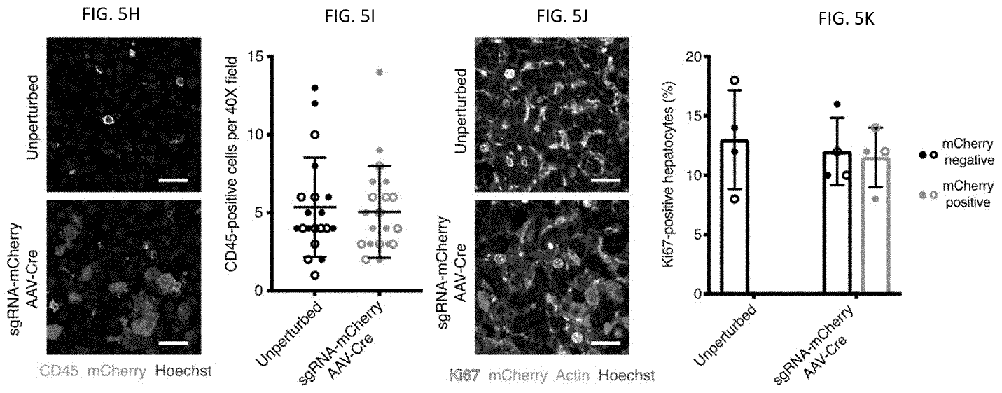

The mouse liver, comprised of tens of millions of hepatocytes, is a promising tissue for genome-wide screening because it provides cell numbers compatible with genome-scale screening in a single mouse. Moreover, given the liver's diverse metabolic functions and impressive regenerative capacity, hepatocytes exhibit a broad range of phenotypes—ranging from those that are universal to all cells to those that are hepatocyte-specific—that are ripe for genetic dissection. Previous attempts at delivering lentivirus to liver have suffered from poor transduction efficiency and immune-mediated clearance of transduced hepatocytes (6, 7). However, the inventors hypothesized that intravenously injecting highly concentrated lentivirus into neonatal mice might avoid these pitfalls and achieve efficient, stable transduction (8). To test this, lentiviruses encoding a non-targeting sgRNA alongside mCherry or mTurq2 reporters were generated and injected at varying doses of an equal mixture of these two lentiviruses into postnatal day (PD) one mice (FIG. 1A). A dose-dependent increase in the percentage of transduced hepatocytes were observed, with a dose of 5×10⁷ transduction units (TU) transducing over 75% of hepatocytes (FIGS. 1B and 1C). Using histologic measurements and the transduction frequency of both fluorescent reporters, it was estimated that a dose of 5×10⁷ TU transduces approximately 10 million hepatocytes per PD1 liver with an average of just two integration events per cell (FIG. 1C and FIG. 5A). This transduction efficiency would afford >200-fold coverage of a 100,000 feature sgRNA library in a single mouse. Importantly, these transduced hepatocytes were distributed uniformly throughout the liver lobule and persisted into adulthood (FIG. 1B and FIGS. 5B and 5C). This lentiviral approach therefore establishes an sgRNA delivery method that is wholly compatible with genome-scale screening in the mouse liver.

It was next asked whether one could use this sgRNA delivery approach as the basis for temporally-controlled protein depletion in hepatocytes. The inventors took advantage of commercially available loxP-stop-loxP-Cas9 (LSL-Cas9) mice in which Cas9 could be induced in nearly all hepatocytes by injecting an adeno-associated virus expressing Cre recombinase from the hepatocyte-specific Tbg promoter (9) (AAV-Cre, FIGS. 5D and 5E). To evaluate the efficiency and kinetics of protein depletion, two long-lived, non-essential proteins were selected: the mitochondrial enzyme MAO-B (encoded by Maob) and the nuclear lamin Lamin B2 (encoded by Lmnb2) (10, 11). After delivering sgMaob-mCherry or sgLmnb2-mCherry lentivirus to PD1 mice, PBS or AAV-Cre was injected at PD5 and livers were harvested at various time points to evaluate protein levels in individual hepatocytes (FIG. 1D). By two weeks after Cas9 induction, MAO-B and Lamin B2 were depleted exclusively in mCherry-positive hepatocytes in mice injected with AAV-Cre (FIGS. 1E and 1F, and FIGS. 5F and 5G). Importantly, this combination of lentiviral-mediated sgRNA delivery, AAV-Cre-mediated induction of Cas9, and resulting gene targeting did not induce detectable liver inflammation nor did it affect hepatocyte turnover (FIG. 5H through 5K). This approach therefore offers an effective platform for hepatocyte-specific protein depletion and genetic screening at any point in the animal's lifetime without obvious collateral perturbation of hepatocyte fitness.

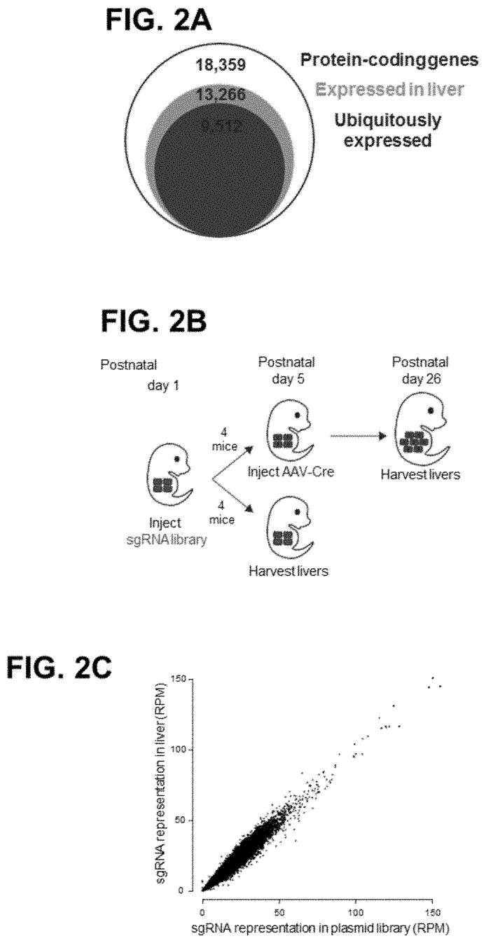

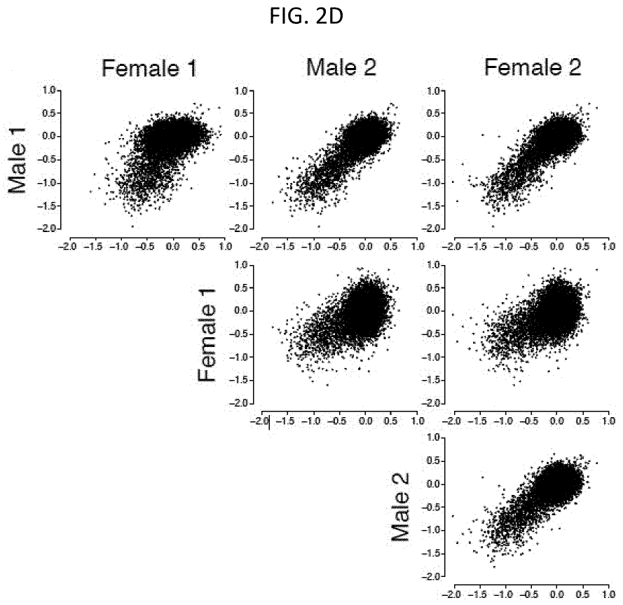

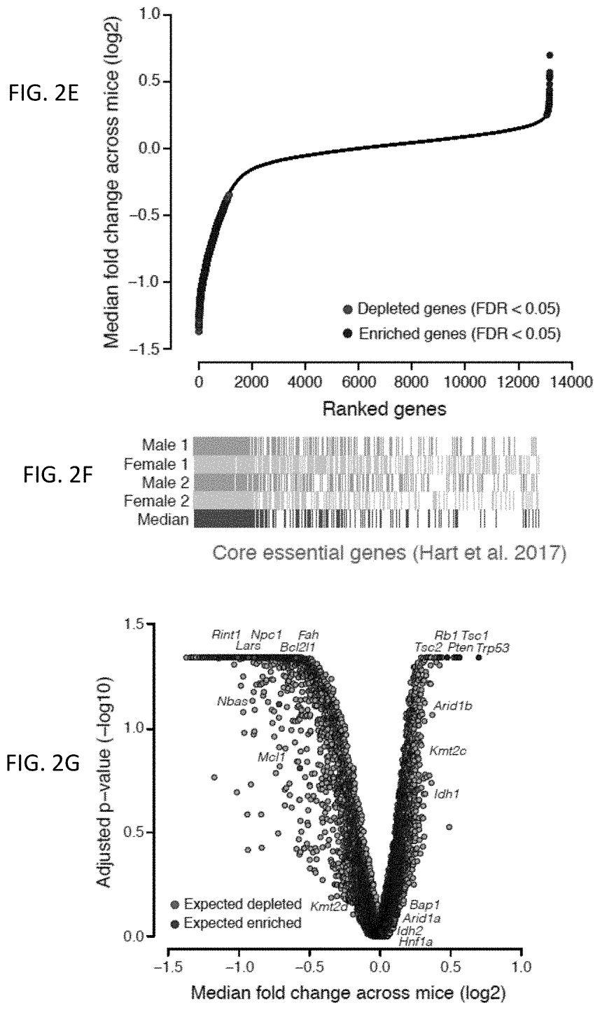

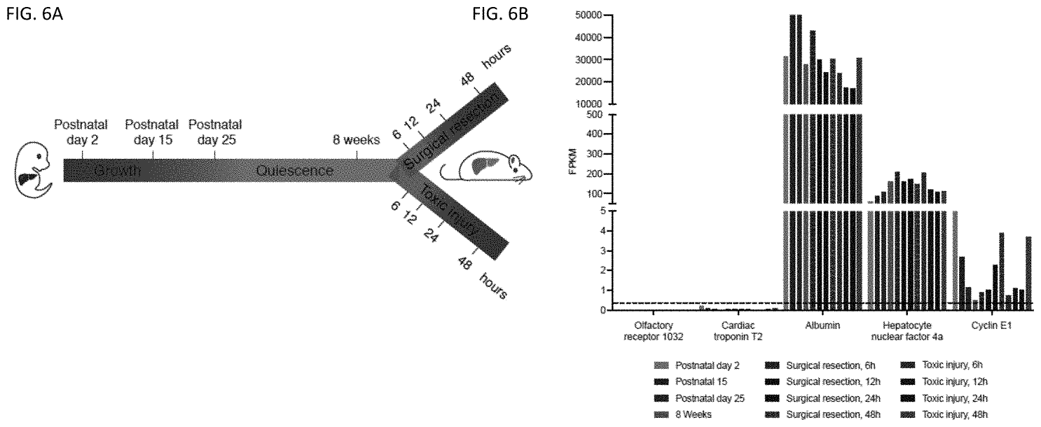

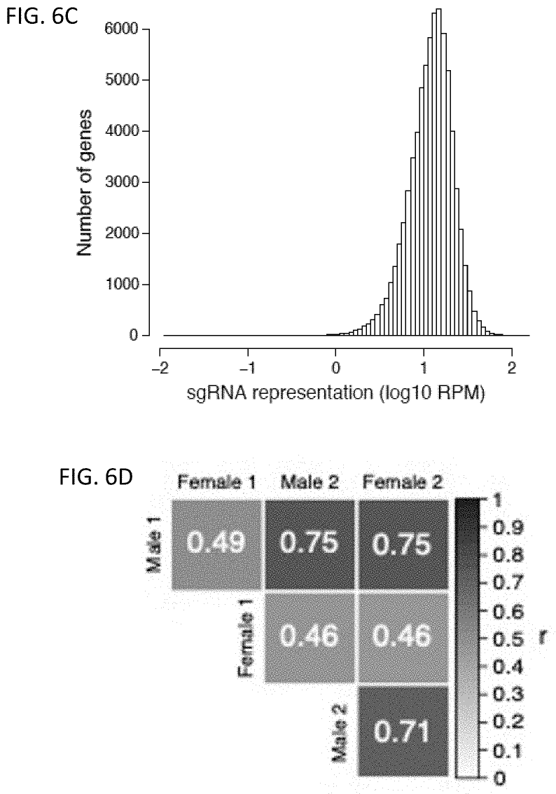

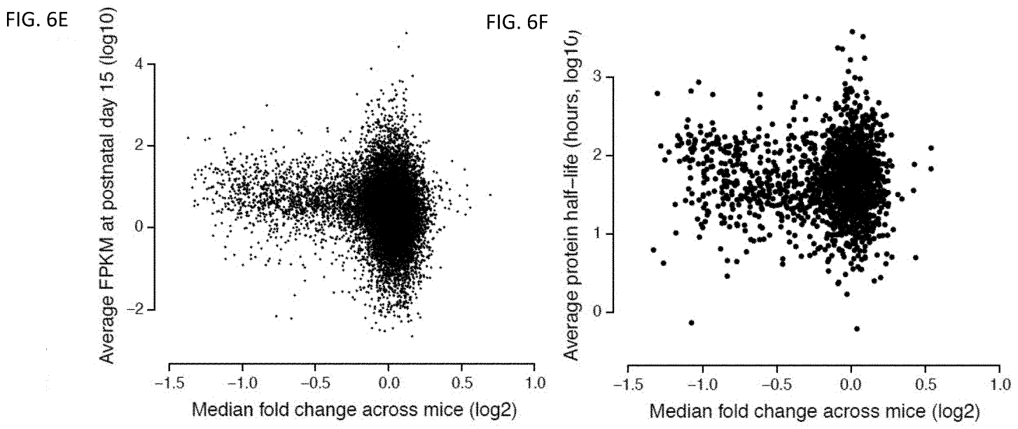

To enable genome-wide screening for diverse hepatocyte phenotypes, an sgRNA library was generated targeting all genes expressed in the developing, quiescent, and regenerating mouse liver. The inventors performed RNA sequencing on livers at various time points during mouse development and after liver injury and determined that 13,266 protein-coding genes were expressed (FPKM>0.3) at one or more time points (FIG. 2A, FIGS. 6A and 6B). The inventors generated an sgRNA library targeting 13,189 of these genes (average of 5 sgRNAs per gene) alongside a previously published set of 6,500 control sgRNAs (˜2,000 non-targeting sgRNAs and ˜4,500 sgRNAs targeting exonic and intronic regions of control genes) for a total of 71,878 unique sgRNAs (12)(FIG. 6C).

With this method in hand, a genome-wide screen for hepatocyte fitness was undertook (FIG. 2B). To screen for the ability of hepatocytes to both persist and proliferate, the inventors elected to screen over a three-week period in neonatal development when hepatocytes undergo approximately three population doublings to increase liver mass. 5×10⁷ TU of our lentiviral library was injected into four female and four male LSL-Cas9 mice at PD1. At PD5, the inventors harvested livers from two males and two females to evaluate the initial library representation. The sgRNA representation in these four livers correlated extremely well (Pearson r=0.97) with the plasmid library and the inventors detected all sgRNAs, indicating effective delivery and recovery of a genome-scale sgRNA library from the neonatal mouse liver (FIG. 2C). In the remaining mice, Cas9 was induced at PD5 and their livers harvested at PD26 to evaluate the final library representation. The Model-based Analysis of Genome-wide CRISPR/Cas9 Knockout (MAGeCK) algorithm was used to identify enriched and depleted genes based on statistical differences in their change in sgRNA abundance at PD26 relative to PD5 (13). Using a false discovery rate cutoff of 0.05, 0-6 significantly enriched and 40-386 significantly depleted genes were identified in individual mice, indicating that this method can detect enriched and depleted genes in a single mouse. The inventors also generated gene-level scores by calculating the median log2 fold change in abundance of sgRNAs targeting a given gene and observed a strong correlation across the four mice (Pearson r=0.46 to 0.75, FIG. 2D, FIG. 6D). To improve the power to detect enriched and depleted genes, the data from all mice was combined and tested as to whether the gene-level scores of each gene deviated significantly from those of all genes across the four mice. Using false discovery rate cutoffs of 0.05 and 0.25, the inventors identified, respectively, 30 and 658 significantly enriched genes and 661 and 1,482 significantly depleted genes across all mice (FIG. 2E). Finally, a unified gene score was calculated representing the median log2 fold change for each gene across all mice. These gene scores were not biased by gene expression level or protein half-life, reaffirming that long-lived proteins were effectively depleted (14) (FIGS. 6E and 6F). Together, these data establish the technical feasibility of genome-wide screening in the mouse liver. Importantly, while screening multiple mice in parallel increases the power to discover significant hits, a single mouse is sufficient to identify significantly enriched and depleted genes.

The inventors next asked whether this screen reliably uncovered regulation of cell fitness. The inventors first confirmed that genes established to be essential in cell culture were highly represented among the depleted genes across mice (15) (FIG. 2F). To evaluate whether this screen could reveal regulation specific to hepatocyte fitness in the liver, the gene scores for two sets of genes known to affect hepatocyte fitness in vivo were assessed: (1) a set of 13 genes established as tumor suppressors in hepatocellular carcinoma (expected to enrich) and (2) a set of seven genes required for hepatocyte viability (expected to deplete) (16). Among the tumor suppressor genes, eight of the 13 genes were significantly enriched (FDR<0.25, FIG. 2G). Among the genes required for hepatocyte viability, all seven genes were significantly depleted (FDR<0.25, FIG. 2G). These results support this screen as a reliable platform for uncovering genetic regulation of hepatocyte fitness in the liver.

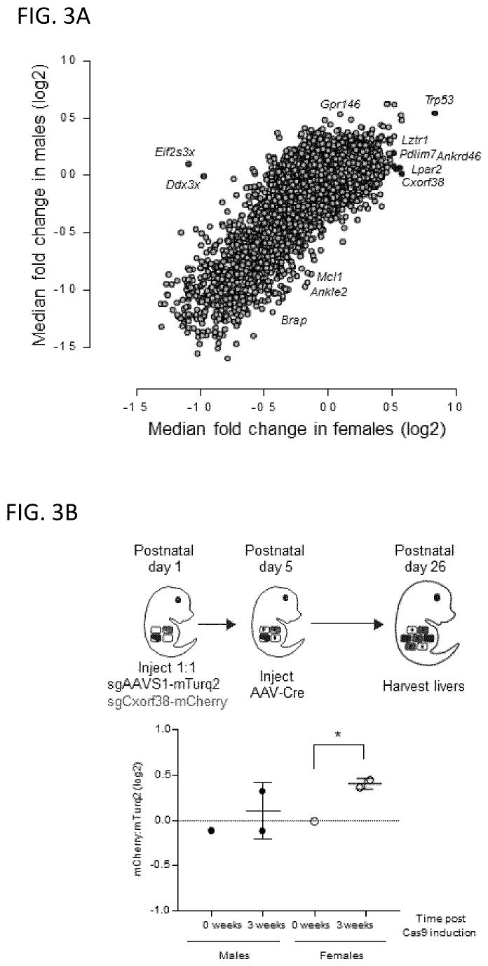

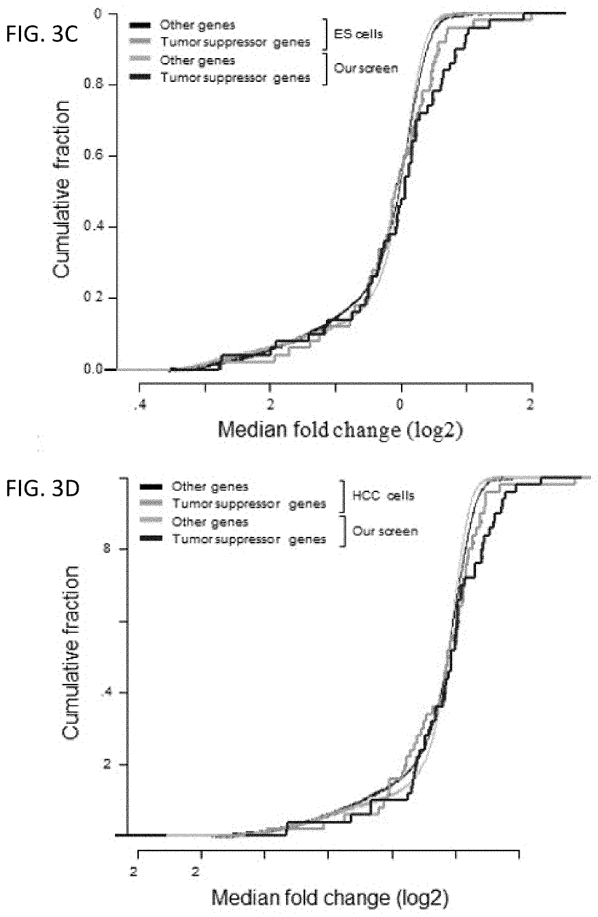

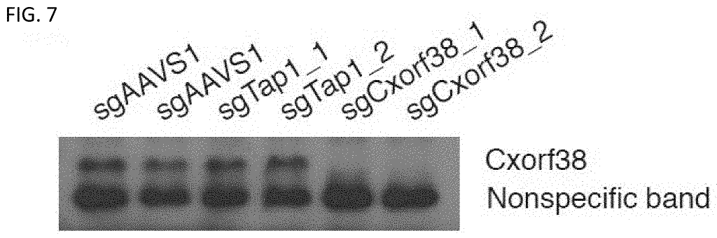

Screening primary cells in their native context has the unique power to capture all organismal regulation of cell fitness, something that cannot be achieved by screening cell lines in culture. One such advantage is the ability to investigate the influence of biological sex in an otherwise isogenic background. To determine whether genes can shape hepatocyte fitness in a sex-dependent manner, the gene scores for all genes in males versus females were compared. Three X-linked genes and nine autosomal genes with sex-specific effects on fitness were identified (FIG. 3A). Two X-linked genes, Ddx3x and Eif2s3x, both involved in protein synthesis, were uniquely essential in females. Both of these genes escape X inactivation and have paralogs on the Y chromosome, Ddx3y and Eif2s3y, with similar function (17). Thus, it is possible that disruption of Ddx3x and Eif2s3x causes a fitness defect in female hepatocytes while male hepatocytes are functionally complemented by the Y chromosome paralogs. Beyond these two genes, the inventors were surprised to identify an uncharacterized open reading frame, 1810030O07Rik, also known as Cxorf38, that when disrupted conferred a fitness advantage exclusively in females. The Cxorf38 gene is also encoded on the X chromosome and has been shown to escape X inactivation but does not have a known Y chromosome paralog (18). It is expressed at low levels across tissues (FPKM ranging from 1-5), with a trend toward higher expression in embryonic versus adult tissues, and is predicted to encode a 320 amino acid protein comprised of a domain of unknown function (19). To confirm that Cxorf38 targeting indeed provides a female-specific fitness advantage, the inventors performed in vivo competition assays to evaluate the fitness of hepatocytes depleted of Cxorf38 relative to control hepatocytes (FIG. 7, FIG. 3B, top panel). Consistent with the screen results, the inventors observed a significant expansion of Cxorf38-depleted hepatocytes relative to control hepatocytes exclusively in female mice (FIG. 3B, bottom panel). The inventors note that cancer genomics efforts have not predicted Cxorf38 to function as a tumor suppressor gene, suggesting that its function might be limited to embryonic and neonatal development (20, 21). The disclosed method thus provides a unique opportunity to investigate how biological sex shapes cellular phenotypes and reveals that a handful of genes regulate hepatocyte fitness in a sex-specific manner

Having established that the majority of genes influence hepatocyte fitness in a sex-independent manner, the inventors next sought to understand broad regulation of hepatocyte fitness by performing gene set enrichment analysis using the unified gene scores across the four mice. No gene sets were identified to be enriched in this screen. However, over 50% of the top 25 most enriched genes in this screen have been established to act as tumor suppressor genes in at least one context (20, 21). Indeed, a set of the top 50 computationally predicted pan-cancer tumor suppressor genes was significantly enriched in this screen (20), something not typically observed in cell culture screens including those in mouse embryonic stem cells or human hepatocellular carcinoma cell lines (22-25) (FIGS. 3C and 3D). One possible explanation for this difference is that cell lines naturally harbor and/or accumulate inactivating mutations in tumor suppressor genes, thereby compromising identification of these genes in subsequent screening (26). The disclosed screen's unique ability to, within only a few population doublings, reliably recover tumor suppressors highlights a key advantage of genome-wide screening in primary tissue—the ability to utilize wild-type cells and thereby identify all genetic perturbations that alter a chosen phenotype.

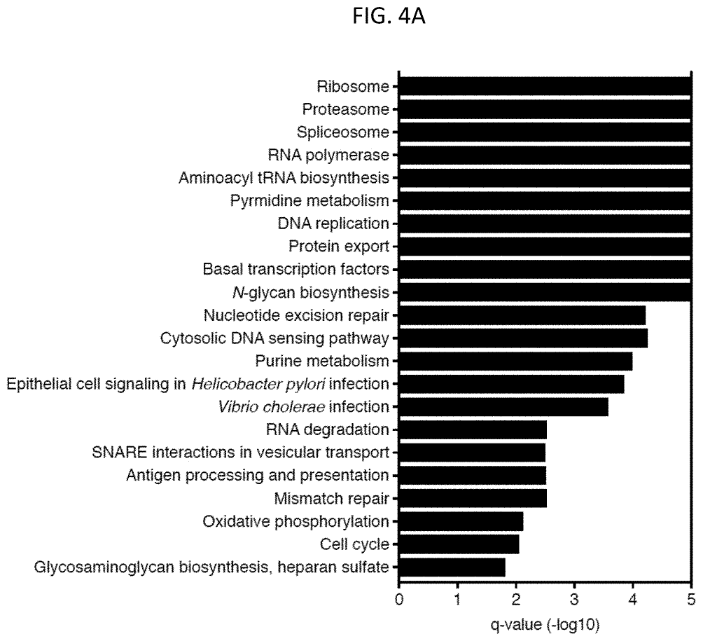

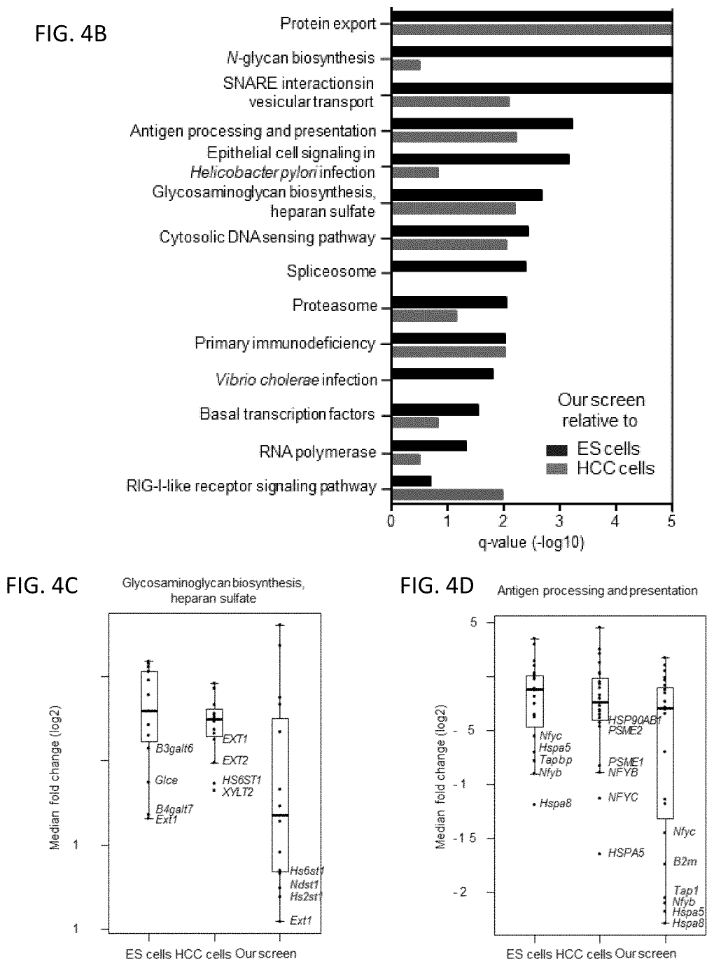

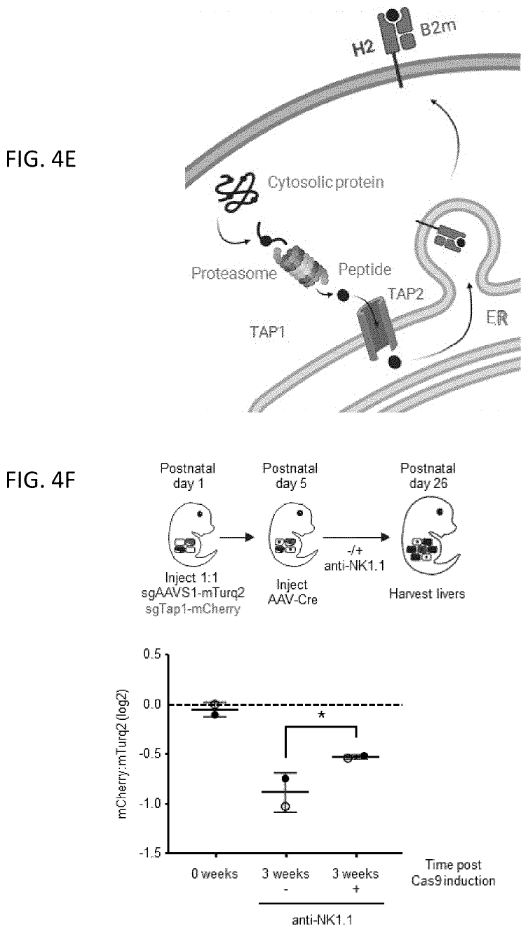

The inventors next focused on genes required for hepatocyte fitness. Several gene sets that were significantly depleted in the screen were identified (FIG. 4A). The gene sets included those previously established as essential for fitness in cell culture, including ribosome, proteasome, spliceosome, and RNA polymerase (1, 2, 11, 15, 24-25). However, several other gene sets not documented to be essential for cells in culture were also identified, including N-glycan biosynthesis, glycosaminoglycan biosynthesis/heparan sulfate, and antigen processing/presentation. Notably, these pathways all play major roles in the presentation or secretion of proteins at the cell surface (27, 28). The screen thus uncovers requirements for cellular fitness shared by cells in culture but indicates additional, possibly cell non-autonomous, requirements for cell fitness in the organismal context.

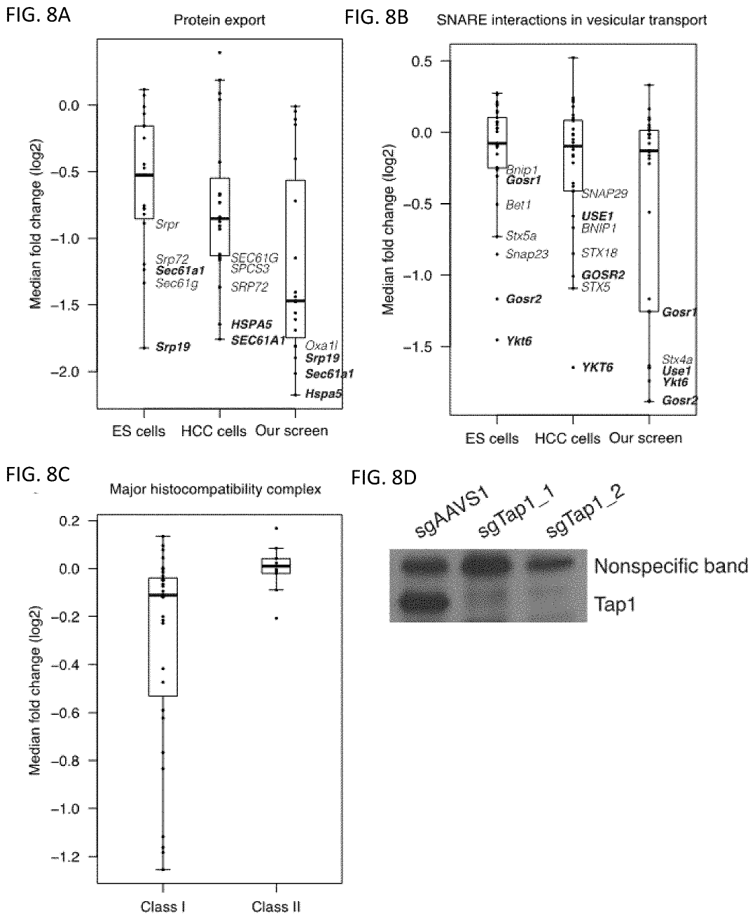

To determine if and how hepatocyte fitness might be influenced by cell non-autonomous factors, the inventors asked whether genes that were preferentially depleted in the screen compared to screens of mouse embryonic stem (ES) cells in culture and human hepatocellular carcinoma (HCC) cell lines in culture (22-25) belonged to any particular gene sets (FIG. 4B). The inventors specifically chose ES cells and HCC cell lines in an effort to control for any species-specific and cell lineage-specific requirements for cell fitness. The inventors focused on four gene sets exhibiting significant depletion in the screen relative to all of the cell culture screens: protein export, SNARE interactions in vesicular transport, antigen processing/presentation, and glycosaminoglycan biosynthesis/heparan sulfate. For the protein export and SNARE interactions gene sets, the most depleted genes in each set were depleted across all screens but to a greater extent in this screen (FIGS. 8A and 8B). However, for the glycosaminoglycan biosynthesis/heparan sulfate and antigen processing/presentation gene sets, some genes were depleted exclusively in this screen (FIGS. 4C and 4D). Within the glycosaminoglycan biosynthesis/heparan sulfate gene set, Hs2st1 and Ndst1 were uniquely depleted in this screen (FIG. 4C). These two genes encode enzymes involved in the biosynthesis of heparan sulfate, a glycosaminoglycan typically conjugated to plasma membrane or extracellular matrix proteins. Heparan sulfate interacts with a variety of extracellular proteins and thereby modulates both mechanotransduction and signal transduction at the cell surface (29). Within the antigen processing/presentation gene set, Tap1 and B2m were uniquely and dramatically depleted in this screen, ranking as the 41^{st} and 320^{th} most depleted genes, respectively (FIG. 4D). Both of these genes are involved in—and required for—presentation of antigens at the cell surface by the class I major histocompatibility complex (MHC) pathway (28). Indeed, within the antigen processing/presentation gene set, eight of the 32 genes attributed to the class I MHC pathway were depleted in this screen whereas none of the 13 genes attributed to the class II MHC pathway exhibited depletion (FDR<0.25, FIG. 8C). This screen thus uncovered two novel requirements for cell fitness—heparan sulfate and class I MHC—that likely exert their functions in a cell non-autonomous manner

The class I MHC pathway presents intracellular antigens at the cell surface. In this pathway, cytoplasmic proteins are degraded by the proteasome, resulting peptides are transported into the endoplasmic reticulum (ER) via a Tap1/Tap2 heterodimer, and these peptides are then loaded onto the class I MHC complex comprised of a heavy chain (H2) and β2-microglobulin (B2m) (FIG. 4E). At the cell surface, class I MHC can interact with both cytotoxic CD8 T cells and natural killer (NK) cells. The latter interaction can provide a pro-survival role by preventing NK cell cytotoxicity. However, loss of class I MHC alone should not be sufficient to induce NK cell cytotoxicity. Classically, NK cell activation requires both a loss of inhibitory signals, via loss of class I MHC, and presence of activating signals, expressed on the surface of stressed or infected cells (30). Although the inventors screening approach involves viral infection of hepatocytes, any inflammation resulting from lentiviral and AAV vectors has been shown to resolve within 72 hours and, consistently, the inventors did not observe any inflammation one week after the combination of lentiviral and AAV-Cre infection (31, 32) (FIGS. 5H and 5I). It was therefore surprising that loss of class I MHC alone would have such a dramatic impact on hepatocyte fitness. To validate this finding, an in vivo competition between cells depleted of Tap1 and control cells was performed (FIG. 8D, FIG. 4F, top panel). Consistent with this screen, hepatocytes depleted of Tap1 were lost over time (FIG. 4F, bottom panel). To test whether this fitness defect occurs via NK cell-mediated cytotoxicity, the inventors repeated the competition in the setting of NK cell depletion and observed reduced loss of Tap1-deficient cells (FIG. 4F). These results confirm an essential role for class I MHC in hepatocyte viability that acts cell non-autonomously via NK cells.

Herein, the inventors successfully performed, to their knowledge, the first genome-wide CRISPR-Cas9 screen for cell fitness in a living organism. In addition to identifying requirements shared by cells in culture, the inventors also uncovered sex-specific and cell non-autonomous regulation of cell fitness. In particular, the inventors have revealed an essential role for class I MHC in hepatocyte viability. The inventors note that expression of class I MHC at the cell surface is exquisitely sensitive to cellular perturbations and that ER stress of various etiologies can impair its expression (33, 34). Moreover, obesity and metabolic syndrome are known to induce ER stress in hepatocytes (35). The inventors speculate that perhaps the rigorous requirement for class I MHC in hepatocytes serves to identify and eliminate stressed cells from the organ and that this surveillance may contribute to the inflammation associated with metabolic syndrome. It will be interesting to determine if this requirement extends to other somatic cell types and whether this serves as a cellular quality control mechanism for cells in an organism.

The disclosed screen's ability to uncover regulation of cell fitness not previously identified in cell culture screens emphasizes the necessity and power of genome-wide screening in the living organism. Importantly, this lentiviral delivery approach coupled with inducible Cas9 can readily be applied to screen diverse hepatocyte phenotypes—from fundamental processes universal to all cells to specialized metabolic and regenerative phenomena unique to hepatocytes—and minimally requires just one mouse. This method can also be adapted to other CRISPR-based approaches including CRISPR interference/activation and Perturb-Seq (36). More broadly, this system establishes the feasibility of genome-wide screening in a living organism and inspires efforts to bring this technology to other organs. Collectively, these diverse applications will bring the experimental tractability once restricted to cell culture to the living organism, enabling unprecedented insight into mammalian physiology and disease.

### REFERENCES

1. O. Shalem, N. E. Sanjana, E. Hartenian, X. Shi, D. A. Scott, T Mikkelson, D. Heckl, B. L. Ebert, D. E. Root, J. G. Doench, F. Zhang, Genome-scale CRISPR-Cas9 knockout screening in human cells, Science 343, 84-87 (2014).

2. T. Wang, J. J. Wei, D. M. Sabatini, E. S. Lander, Genetic screens in human cells using the CRISPR-Cas9 system, Science 343, 80-84 (2014).

3. C. Xu, X. Qi, X. Du, H. Zou, F. Gao, T. Feng, H. Lu, S. Li, X. An, L. Zhang, Y. Wu, Y. Liu, N. Li, M. R. Capecchi, S. Wu, piggyBac mediates efficient in vivo CRISPR library screening for tumorigenesis in mice, Proc. Natl. Acad. Sci. U.S.A. 114, 722-727 (2017).

4. S. Chen, N. E. Sanjana, K. Zheng, O. Shalem, K. Lee, X. Shi, D. A. Scott, J. Song, J. Q. Pan, R. Weissleder, H. Lee, F. Zhang, P. A. Sharp, Genome-wide CRISPR Screen in a Mouse Model of Tumor Growth and Metastasis, Cell 160, 1246-1260 (2015).

5. M. B. Dong, G. Wang, R. D. Chow, L. Ye, L. Zhu, X. Dai, J. J. Park, H. R. Kim, Y. Errami, C. D. Guzman, X. Zhou, K. Y. Chen, P. A. Renauer, Y. Du, J. Shen, S. Z. Lam, J. J. Zhou, D. R. Lannin, R. S. Herbst, S. Chen, Systematic Immunotherapy Target Discovery Using Genome-Scale In Vivo CRISPR Screens in CD8 T Cells, Cell 178, 1189-1204.e23 (2019).

6. A. Pfeifer, T. Kessler, M. Yang, E. Baranov, N. Kootstra, D. A. Cheresh, R. M. Hoffman, I. M. Verma, Transduction of liver cells by lentiviral vectors: analysis in living animals by fluorescence imaging, Molecular Therapy 3, 319-322 (2001).

7. S. N. Waddington, K. A. Mitrophanous, F. M. Ellard, S. M. K. Buckley, M. Nivsarkar, L. Lawrence, H. T. Cook, F. Al-Allaf, B. Bigger, S. M. Kingsman, C. Coutelle, M. Themis, Long-term transgene expression by administration of a lentivirus-based vector to the fetal circulation of immuno-competent mice, Gene Ther. 10, 1234-1240 (2003).

8. T. H. Nguyen, M. Bellodi-Privato, D. Aubert, V. Pichard, A. Myara, D. Trono, N. Ferry, Therapeutic lentivirus-mediated neonatal in vivo gene therapy in hyperbilirubinemic Gunn rats, Molecular Therapy 12, 852-859 (2005).

9. R. J. Platt, S. Chen, Y. Zhou, M. J. Yim, L. Swiech, H. R. Kempton, J. E. Dahlman, O. Parnas, T. M. Eisenhaure, M. Jovanovic, D. B. Graham, S. Jhunjhunwala, M. Heidenreich, R. J. Xavier, R. Langer, D. G. Anderson, N. Hacohen, A. Regev, G. Feng, P. A. Sharp, F. Zhang, CRISPR-Cas9 knockin mice for genome editing and cancer modeling, Cell 159, 440-455 (2014).

10. T. Mathieson, H. Franken, J. Kosinski, N. Kurzawa, N. Zinn, G. Sweetman, D. Poeckel, V. S. Ratnu, M. Schramm, I. Becher, M. Steidel, K.-M. Noh, G. Bergamini, M. Beck, M. Bantscheff, M. M. Savitski, Systematic analysis of protein turnover in primary cells, Nat Commun 9, 689 (2018).

11. T. Wang, K. Birsoy, N. W. Hughes, K. M. Krupczak, Y. Post, J. J. Wei, E. S. Lander, D. M. Sabatini, Identification and characterization of essential genes in the human genome, Science 350, 1096-1101 (2015).

12. J. G. Doench, E. Hartenian, D. B. Graham, Z. Tothova, M. Hegde, I. Smith, M. Sullender, B. L. Ebert, R. J. Xavier, D. E. Root, Rational design of highly active sgRNAs for CRISPR-Cas9-mediated gene inactivation, Nat Biotech 32, 1262-1267 (2014).

13. W. Li, H. Xu, T. Xiao, L. Cong, M. I. Love, F. Zhang, R. A. Irizarry, J. S. Liu, M. Brown, X. S. Liu, MAGeCK enables robust identification of essential genes from genome-scale CRISPR/Cas9 knockout screens, Genome Biol 15, 554 (2014).

14. B. Schwanhäusser, D. Busse, N. Li, G. Dittmar, J. Schuchhardt, J. Wolf, W. Chen, M. Selbach, Global quantification of mammalian gene expression control, Nature 473, 337-342 (2011).

15. T. Hart, A. H. Y. Tong, K. Chan, J. Van Leeuwen, A. Seetharaman, M. Aregger, M. Chandrashekhar, N. Hustedt, S. Seth, A. Noonan, A. Habsid, O. Sizova, L. Nedyalkova, R. Climie, L. Tworzyanski, K. Lawson, M. A. Sartori, S. Alibeh, D. Tieu, S. Masud, P. Mero, A. Weiss, K. R. Brown, M. Usaj, M. Billmann, M. Rahman, M. Constanzo, C. L. Myers, B. J. Andrews, C. Boone, D. Durocher, J. Moffat, Evaluation and Design of Genome-Wide CRISPR/SpCas9 Knockout Screens, G3 (Bethesda) 7, 2719-2727 (2017).

16. Cancer Genome Atlas Research Network. Electronic address: wheeler@bcm.edu, Cancer Genome Atlas Research Network, G. Saksena, Comprehensive and Integrative Genomic Characterization of Hepatocellular Carcinoma, Cell 169, 1327-1341.e23 (2017).

17. J. B. Berletch, W. Ma, F. Yang, J. Shendure, W. S. Noble, C. M. Disteche, X. Deng, M. S. Bartolomei, Ed. Escape from X Inactivation Varies in Mouse Tissues, PLoS Genet 11, e1005079-26 (2015).

18. Y. Zhang, K. Klein, A. Sugathan, N. Nassery, A. Dombkowski, U. M. Zanger, D. J. Waxman, Transcriptional profiling of human liver identifies sex-biased genes associated with polygenic dyslipidemia and coronary artery disease, PLoS ONE 6, e23506 (2011).

19. F. Yue, Y. Cheng, A. Breschi, J. Vierstra, W. Wu, T. Ryba, R. Sandstrom, Z. Ma, C. Davis, B. D. Pope, Y. Shen, D. D. Pervouchine, S. Djebali, R. E. Thurman, R. Kaul, E. Rynes, A. Kirilusha, G. K. Marinov, B. A. Williams, D. Trout, H. Amrhein, K. Fisher-Aylor, I. Antoshechkin, G. DeSalvo, L.-H. See, M. Fastuca, J. Drenkow, C. Zaleski, A. Dobin, P. Prieto, J. Lagarde, G. Bussotti, A. Tanzer, O. Denas, K. Li, M. A. Bender, M. Zhang, R. Byron, M. T. Groudine, D. McCleary, L. Pham, Z. Ye, S. Kuan, L. Edsall, Y.-C. Wu, M. D. Rasmussen, M. S. Bansal, M. Kellis, C. A. Keller, C. S. Morrissey, T. Mishra, D. Jain, N. Dogan, R. S. Harris, P. Cayting, T. Kawli, A. P. Boyle, G. Euskirchen, A. Kundaje, S. Lin, Y. Lin, C. Jansen, V. S. Malladi, M. S. Cline, D. T. Erickson, V. M. Kirkup, K. Learned, C. A. Sloan, K. R. Rosenbloom, B. Lacerda de Sousa, K. Beal, M. Pignatelli, P. Flicek, J. Lian, T. Kahveci, D. Lee, W. J. Kent, M. Ramalho Santos, J. Herrero, C. Notredame, A. Johnson, S. Vong, K. Lee, D. Bates, F. Neri, M. Diegel, T. Canfield, P. J. Sabo, M. S. Wilken, T. A. Reh, E. Giste, A. Shafer, T. Kutyavin, E. Haugen, D. Dunn, A. P. Reynolds, S. Neph, R. Humbert, R. S. Hansen, M. De Bruijn, L. Selleri, A. Rudensky, S. Josefowicz, R. Samstein, E. E. Eichler, S. H. Orkin, D. Levasseur, T. Papayannopoulou, K.-H. Chang, A. Skoultchi, S. Gosh, C. Disteche, P. Treuting, Y. Wang, M. J. Weiss, G. A. Blobel, X. Cao, S. Zhong, T. Wang, P. J. Good, R. F. Lowdon, L. B. Adams, X.-Q. Zhou, M. J. Pazin, E. A. Feingold, B. Wold, J. Taylor, A. Mortazavi, S. M. Weissman, J. A. Stamatoyannopoulos, M. P. Snyder, R. Guigó, T. R. Gingeras, D. M. Gilbert, R. C. Hardison, M. A. Beer, B. Ren, Mouse ENCODE Consortium, A comparative encyclopedia of DNA elements in the mouse genome, Nature 515, 355-364 (2014).

20. T. Davoli, A. W. Xu, K. E. Mengwasser, L. M. Sack, J. C. Yoon, P. J. Park, S. J. Elledge, Cumulative Haploinsufficiency and Triplosensitivity Drive Aneuploidy Patterns and Shape the Cancer Genome, Cell 155, 948-962 (2013).

21. J. G. Tate, S. Bamford, H. C. Jubb, Z. Sondka, D. M. Beare, N. Bindal, H. Boutselakis, C. G. Cole, C. Creatore, E. Dawson, P. Fish, B. Harsha, C. Hathaway, S. C. Jupe, C. Y. Kok, K. Noble, L. Ponting, C. C. Ramshaw, C. E. Rye, H. E. Speedy, R. Stefancsik, S. L. Thompson, S. Wang, S. Ward, P. J. Campbell, S. A. Forbes, COSMIC: the Catalogue Of Somatic Mutations In Cancer, Nucleic Acids Research 47, D941-D947 (2019).

22. S. Shohat, S. Shifman, Genes essential for embryonic stem cells are associated with neurodevelopmental disorders, Genome Research 29, 1910-1918 (2019).

23. K. Tzelepis, H. Koike-Yusa, E. De Braekeleer, Y. Li, E. Metzakopian, O. M. Dovey, A. Mupo, V. Grinkevich, M. Li, M. Mazan, M. Gozdecka, S. Ohnishi, J. Cooper, M. Patel, T. McKerrell, B. Chen, A. F. Domingues, P. Gallipoli, S. Teichmann, H. Ponstingl, U. McDermott, J. Saez-Rodriguez, B. J. P. Huntly, F. Iorio, C. Pina, G. S. Vassiliou, K. Yusa, A CRISPR Dropout Screen Identifies Genetic Vulnerabilities and Therapeutic Targets in Acute Myeloid Leukemia, CellReports 17, 1193-1205 (2016).

24. R. M. Meyers, J. G. Bryan, J. M. McFarland, B. A. Weir, A. E. Sizemore, H. Xu, N. V. Dharia, P. G. Montgomery, G. S. Cowley, S. Pantel, A. Goodale, Y. Lee, L. D. Ali, G. Jiang, R. Lubonja, W. F. Harrington, M. Strickland, T. Wu, D. C. Hawes, V. A. Zhivich, M. R. Wyatt, Z. Kalani, J. J. Chang, M Okamoto, K. Stegmaier, T. R. Golub, J. S. Boehm, F. Vazquez, D. E. Root, W. C. Hahn, A. Tsherniak, Computational correction of copy number effect improves specificity of CRISPR-Cas9 essentiality screens in cancer cells, Nature Genetics 49, 1779-1784 (2017).

25. J. M. Dempster, J. Rossen, M. Kazachkova, J. Pan, G. Kugener, D. E. Root, A. Tsherniak, Extracting Biological Insights from the Project Achilles Genome-Scale CRISPR Screens in Cancer Cell Lines, bioRxiv 20, 21-35 (2020).

26. F. T. Merkle, S. Ghosh, N. Kamitaki, J. Mitchell, Y. Avior, C. Mello, S. Kashin, S. Mekhoubad, D. Ilic, M. Charlton, G. Saphier, R. E. Handsaker, G. Genovese, S. Bar, N. Benvenisty, S. A. McCarroll, K. Eggan, Human pluripotent stem cells recurrently acquire and expand dominant negative P53 mutations, Nature 545, 229-233 (2017).

27. N. Cherepanova, S. Shrimal, R. Gilmore, N-linked glycosylation and homeostasis of the endoplasmic reticulum, Curr. Opin. Cell Biol. 41, 57-65 (2016).

28. D. R. Peaper, P. Cresswell, Regulation of MHC class I assembly and peptide binding, Annu. Rev. Cell Dev. Biol. 24, 343-368 (2008).

29. M. Ravikumar, R. A. A. Smith, V. Nurcombe, S. M. Cool, Heparan Sulfate Proteoglycans: Key Mediators of Stem Cell Function, Front Cell Dev Biol 8, 581213 (2020).

30. C. J. Chan, M. J. Smyth, L. Martinet, Molecular mechanisms of natural killer cell activation in response to cellular stress, Cell Death Differ 21, 5-14 (2014).

31. B. D. Brown, G. Sitia, A. Annoni, E. Hauben, L. S. Sergi, A. Zingale, M. G. Roncarolo, L. G. Guidotti, L. Naldini, In vivo administration of lentiviral vectors triggers a type I interferon response that restricts hepatocyte gene transfer and promotes vector clearance, Blood 109, 2797-2805 (2007).

32. R. W. H. Arun Srivastava, Innate immune responses to AAV vectors, 1-10 (2011).

33. S. F. de Almeida, J. V. Fleming, J. E. Azevedo, M. Carmo-Fonseca, M. de Sousa, Stimulation of an unfolded protein response impairs MHC class I expression, The Journal of Immunology 178, 3612-3619 (2007).

34. D. P. Granados, P.-L. Tanguay, M.-P. Hardy, E. Caron, D. de Verteuil, S. Meloche, C. Perreault, ER stress affects processing of MHC class I-associated peptides, BMC Immunol 10, 10-15 (2009).

35. Y. Ogawa, K. Imajo, Y. Honda, T. Kessoku, W. Tomeno, S. Kato, K. Fujita, M. Yoneda, S. Saito, Y. Saigusa, H. Hyogo, Y. Sumida, Y. Itoh, K. Eguchi, T. Yamanaka, K. Wada, A. Nakajima, Palmitate-induced lipotoxicity is crucial for the pathogenesis of nonalcoholic fatty liver disease in cooperation with gut-derived endotoxin, Sci. Rep., 1-14 (2018).

36. A. Pickar-Oliver, C. A. Gersbach, The next generation of CRISPR-Cas technologies and applications, Nat Rev Mol Cell Biol 20, 490-507 (2019).

37. M. K. Chuah, I. Petrus, P. De Bleser, C. Le Guiner, G. Gernoux, O. Adjali, N. Nair, J. Willems, H. Evens, M. Y. Rincon, J. Matrai, M. Di Matteo, E Samara-Kuko, B. Yan, A. Acosta-Sanchez, A. Meliani, G. Cherel, V. Blouin, O. Christophe, P. Moullier, F. Mingozzi, T. VandenDriessche, Liver-specific transcriptional modules identified by genome-wide in silico analysis enable efficient gene therapy in mice and non-human primates, Mol. Ther. 22, 1605-1613 (2014).

38. C. P. Fulco, M. Munschauer, R. Anyoha, G. Munson, S. R. Grossman, E. M. Perez, M. Kane, B. Cleary, E. S. Lander, J. M. Engreitz, Systematic mapping of functional enhancer-promoter connections with CRISPR interference, Science 354, 769-773 (2016).

39. B. Chen, L. A. Gilbert, B. A. Cimini, J. Schnitzbauer, W. Zhang, G.-W. Li, J. Park, E. H. Blackburn, J. S. Weissman, L. S. Qi, B. Huang, Dynamic imaging of genomic loci in living human cells by an optimized CRISPR/Cas system, Cell 155, 1479-1491 (2013).

40. N. E. Sanjana, O. Shalem, F. Zhang, Improved vectors and genome-wide libraries for CRISPR screening, Nat Meth 11, 783-784 (2014).

41. K. A. Knouse, K. E. Lopez, M. Bachofner, A. Amon, Chromosome Segregation Fidelity in Epithelia Requires Tissue Architecture, Cell 175, 200-211.e13 (2018).

42. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner, Bioinformatics 29, 15-21 (2013).

43. Y. Liao, G. K. Smyth, W. Shi, featureCounts: an efficient general purpose program for assigning sequence reads to genomic features, Bioinformatics 30, 923-930 (2014).

44. M. I. Love, W. Huber, S. Anders, Moderated estimation of fold change and dispersion for RNA-seq data with DESeq2, Genome Biol 15, 550 (2014).

45. J. G. Doench, N. Fusi, M. Sullender, M. Hegde, E. W. Vaimberg, K. F. Donovan, I. Smith, Z. Tothova, C. Wilen, R. Orchard, H. W. Virgin, J. Listgarten, D. E. Root, Optimized sgRNA design to maximize activity and minimize off-target effects of CRISPR-Cas9, Nat Biotech 34, 184-191 (2016).

Materials and Methods

Animals

C57BL/6J mice (strain 000664) and LSL-Cas9 mice (strain 026175) were purchased from the Jackson Laboratory. Mice were either singly- or group-housed with a 12-hour light-dark cycle (light from 7 AM to 7 PM, dark from 7 PM to 7 AM) in a specific-pathogen-free animal facility with unlimited access to food and water. To deliver lentivirus, up to 100 μL of lentivirus in PBS was injected into the temporal vein of postnatal day one mice. For protein depletion tests, mice were injected with 1.25×10⁷ transduction units (TU) of sgRNA-mCherry lentivirus. For the screen, mice were injected with 5×10⁷ TU of sgRNA-mCherry lentiviral library. For validating Cxorf38 and Tap1, mice were injected with 1×10⁷ TU of an equal mixture of sgAAVS1-mTurq2 and sgCxorf38-mCherry or sgTap1-mCherry lentiviruses. To deliver AAV-Cre, a stock solution of AAV8-TBG-Cre (Addgene 107787-AAV8) was diluted in PBS to a total volume of 20 μL and injected intraperitoneally into postnatal day five mice. For protein depletion tests, the screen, and validating hits, mice were injected with 2×10⁷ GC of AAV-TBG-Cre. To deplete NK cells, 15 μg of anti-NK1.1 (clone PK136, BioXCell) was injected intraperitoneally every three days beginning at postnatal day five. All animal procedures were approved by the Massachusetts Institute of Technology Committee on Animal Care.

Cell Lines

The mouse hepatocyte cell line AML12 was purchased from the American Type Culture Collection (ATCC) and cultured in DMEM/F12 medium supplemented with 10% fetal bovine serum, 10 μg/mL insulin, 5.5 μg/mL transferrin, 5 ng/mL selenium, and 40 ng/mL dexamethasone (ThermoFisher Scientific). HEK-293T cells were cultured in DMEM supplemented with 10% fetal bovine serum, 100 units/mL penicillin, and 100 μg/mL streptomycin (ThermoFisher Scientific).

Vector Construction

The vector was produced through the following steps: 1) removal of the EFS-NS promoter and Cas9 from the parental vector and insertion of a hepatocyte-specific promoter driving dsRed expression, 2) replacement of dsRed with mCherry or mTurq2, and 3) removal of the puromycin resistance cassette.

To produce pLCv2-opti-stuffer-dsRed-puro, 100 ng of a synthetic gblock encoding the HS-CRM8-TTRmin module (37) upstream of dsRed (Integrated DNA Technologies) and 1 μg of sgOpti (gift from Eric Lander and David Sabatini, Addgene plasmid #85681) (38), a lentiCRISPRv2 derivative containing an optimized scaffold (5′-GTTTAAGAGCTATGCTGGAAACAGCATAGCAAGTTT-3′ (SEQ ID NO: 1)) (39) were digested sequentially with NheI and BamHI (New England Biolabs). The vector and fragment were purified using the QIAquick Gel Extraction Kit (Qiagen) and ligated with T4 DNA Ligase (New England Biolabs) in an 11 μL reaction to replace the EFS-NS promoter and Cas9 with the gblock fragment. 2.5 μL of the ligation was used to transform Stbl2 cells (Invitrogen) and DNA was isolated from ampicillin-resistant colonies with the QIAprep Spin Miniprep Kit (Qiagen). Clones were verified by Sanger sequencing (Quintara Biosciences) prior to retransformation and maxiprep using the ZymoPURE II Plasmid Maxiprep Kit (Zymo Research).

HS-CRM8-TTRmin-dsRed:
(SEQ ID NO: 2)
GAATTCGCTAGCACCGGCGCGCCGGGGGAGGCTGCTGGTGAATATTAACC
AAGGTCACCCCAGTTATCGGAGGAGCAAACAGGGGCTAAGTCCACACGCG
TGGTACCGTCTGTCTGCACATTTCGTAGAGCGAGTGTTCCGATACTCTAA
TCTCCCTAGGCAAGGTTCATATTTGTGTAGGTTACTTATTCTCCTTTTGT
TGACTAAGTCAATAATCAGAATCAGCAGGTTTGGAGTCAGCTTGGCAGGG
ATCAGCAGCCTGGGTTGGAAGGAGGGGGTATAAAAGCCCCTTCACCAGGA
GAAGCCGTCACACAGATCCACAAGCTCCTGACCGGTTCTAGAGCGCTGCC
ACCATGGTGCGCTCCTCCAAGAACGTCATCAAGGAGTTCATGCGCTTCAA
GGTGCGCATGGAGGGCACCGTGAACGGCCACGAGTTCGAGATCGAGGGCG
AGGGCGAGGGCCGCCCCTACGAGGGCCACAACACCGTGAAACTGAAGGTG
ACCAAGGGCGGCCCCCTGCCCTTCGCCTGGGACATCCTGTCCCCCCAGTT
CCAGTACGGCTCCAAGGTGTACGTGAAGCACCCCGCCGACATCCCCGACT
ACAAGAAGCTGTCCTTCCCCGAGGGCTTCAAGTGGGAGCGCGTGATGAAC
TTCGAGGACGGCGGCGTGGTGACCGTGACCCAAGACTCCTCCCTGCAGGA
CGGCTGCTTCATCTACAAGGTGAAGTTCATCGGCGTGAACTTCCCCTCCG
ACGGCCCCGTAATGCAGAAGAAGACCATGGGCTGGGAGGCCTCCACCGAG
CGCCTGTACCCCCGCGACGGCGTGCTGAAGGGCGAAATCCACAAGGCCCT
GAAGCTGAAGGACGGCGGCCACTACCTGGTGGAGTTCAAGTCCATCTACA
TGGCCAAGAAGCCCGTGCAGCTGCCCGGCTACTACTACGTGGACTCCAAG
CTGGACATCACCTCCCACAACGAGGACTACACCATCGTGGAGCAGTACGA
GCGCACCGAAGGCCGCCACCACCTGTTCCTGGGATCCGGCGCAACAAACT
TCTCTCTGCTGAAACAAGCCGGAGATGTCGAAGAGAATCCTGGACCGACC
GAG

To construct pLCv2-opti-stuffer-mCherry-puro and pLCv2-opti-stuffer-mTurq2-puro, mCherry and mTurq2 were amplified from pKL028 and mTurquoise2-CMV (gifts from Iain Cheeseman), respectively, for 25 cycles with Q5 HotStart Polymerase (New England Biolabs) using the following primers (underlined nucleotides are homologous to the vector):

pLC_EBFP2_F:
(SEQ ID NO: 3)
GGTTCTAGAGCGCTGCCACCATGGTGAGCAAGGGCGAGGAG
pLC_EBFP2_R:
(SEQ ID NO: 4)
GCCGGATCCCTTGTACAGCTCGTCCATGCC

Amplicons and pLCv2-opti-stuffer-dsRed-puro were digested with XbaI and BamHI HF (New England Biolabs) and purified, ligated, transformed, and DNA was isolated and sequence verified as above.

To construct pLCv2-opti-stuffer-mCherry and pLCv2-opti-stuffer-mTurq2, a fragment encompassing the WPRE and 3′LTR was amplified from pLCv2-opti-stuffer-mCherry-puro as above using the following primers:

Puro_removal_F:
(SEQ ID NO: 5)
TGAACGCGTTAAGTCGACAATCAACC
Puro_removal_R:
(SEQ ID NO: 6)
TCGAGGCTGATCAGCGGGTTTAAAC

The amplicon and pLCv2-opti-stuffer-mCherry-puro (or -mTurq2-puro) were digested with BsrGI-HF and PmeI (New England Biolabs) and purified as above. NEBuilder HiFi DNA Assembly Master Mix (New England Biolabs) was used to assemble 25 ng each of vector and fragment in a 20 μL reaction for 15 min at 50° C. 50 μL of DH5-alpha cells were transformed with 2 μL assembly mix, and DNA was isolated and sequence verified as described above.

Individual sgRNAs were cloned as previously described (40), using the following oligonucleotides (Integrated DNA Technologies):

sgAAVS1_F:
(SEQ ID NO: 7)
CACCGGGGCCACTAGGGACAGGAT
sgAAVS1_R:
(SEQ ID NO: 8)
AAACATCCTGTCCCTAGTGGCCCC
sgMaob_1_F:
(SEQ ID NO: 9)
CACCGACGGATAAAGGATATACTTG
sgMaob_1_R:
(SEQ ID NO: 10)
AAACCAAGTATATCCTTTATCCGTC
sgMaob_2_F:
(SEQ ID NO: 11)
CACCGGGAAAATCATATGCCTTCAG
sgMaob_2_R:
(SEQ ID NO: 12)
AAACCTGAAGGCATATGATTTTCCC
sgLmnb2_1_F:
(SEQ ID NO: 13)
CACCGAGGTACGGGAGACCCGACGG
sgLmnb2_1_R:
(SEQ ID NO: 14)
AAACCCGTCGGGTCTCCCGTACCTC
sgLmnb2_2_F:
(SEQ ID NO: 15)
CACCGCTGCGCACCTACCTCACCGT
sgLmnb2_2_R:
(SEQ ID NO: 16)
AAACACGGTGAGGTAGGTGCGCAGC
sgCxorf38_1_F:
(SEQ ID NO: 17)
CACCGGTCAACCACAAAGTGATCAC
sgCxorf38_1_R:
(SEQ ID NO: 18)
AAACGTGATCACTTTGTGGTTGACC
sgCxorf38_2_F:
(SEQ ID NO: 19)
CACCGTCTGCGCATACCTGACACTG
sgCxorf38_2_R:
(SEQ ID NO: 20)
AAACCAGTGTCAGGTATGCGCAGAC
sgTap1_1_F:
(SEQ ID NO: 21)
CACCGGGTGCCAACGAGCCACTGAG
sgTap1_1_R:
(SEQ ID NO: 22)
AAACCTCAGTGGCTCGTTGGCACCC
sgTap1_2_F:
(SEQ ID NO: 23)
CACCGGGTAGAGAACGAATGAGACA
sgTap1_2_R:
(SEQ ID NO: 24)
AAACTGTCTCATTCGTTCTCTACCC

Lentivirus Preparation, Titration, and Concentration

HEK-293T cells were seeded at a density of 750,000 cells/mL in 20 mL viral production medium (IMDM supplemented with 20% inactivated fetal serum, Thermo Fisher Scientific). After 24 hours, media was changed to fresh viral production medium. At 32 hours post-seeding, cells were transfected with a mix containing 76.8 μL Xtremegene-9 transfection reagent (Thermo Fisher Scientific), 3.62 μg pCMV-VSV-G (gift from Bob Weinberg, Addgene plasmid #8454), 8.28 μg psPAX2 (gift from Didier Trono, Addgene plasmid #12260), and 20 μg sgRNA plasmid in Opti-MEM (Thermo Fisher Scientific) to a final volume of 1 mL. Media was changed 16 hours later to fresh viral production medium. At 48 hours after transfection, virus was collected and filtered through a 0.45 μm filter, aliquoted, and stored at −80° C. until use.

To determine lentivirus titer, AML12 cells were transduced with a dilution series of lentivirus in the presence of 10 μg/mL polybrene for 16 hours. After four days, cells were harvested for flow cytometry analysis to determine percent of mTurq2- or mCherry-positive cells. To concentrate lentivirus, lentiviral supernatant was ultracentrifuged at 23,000 RPM at 4° C. for 2 hours in an SW 32 Ti swinging bucket rotor (Beckman Coulter). After centrifugation, media was decanted and pellets were air-dried at room temperature for 15 minutes. Pellets were then resuspended in PBS at room temperature for 30 minutes with gentle trituration. Concentrated lentivirus in PBS was stored for up to one week at 4° C. prior to injection into mice.

sgRNA Validation

To validate individual sgRNA sequences for the ability to deplete target proteins, AML12 cells stably expressing Cas9-EGFP were transduced with sgAAVS1-mTurq2 or sgRNA-mCherry lentiviruses as described for lentivirus titration. After four days, transduced cells were subjected to fluorescence activated cell sorting to purify cells expressing both EGFP and mTurq2 or EGFP and mCherry and cultured for another seven days. Cells were then harvested to prepare protein lysates and perform immunoblotting. To validate sgRNAs targeting Tap1, cells were treated with 10 ng/mL of IFNγ 20 hours prior to harvest to upregulate Tap1 expression such that it could be detected by immunoblotting.

Immunostaining

Livers were harvested and fixed in 4% paraformaldehyde in PBS at room temperature for 16-24 hours. Tissues were then washed with PBS and frozen in O.C.T. Compound (Tissue-Tek). Tissue sections of 12 to 30 μm thickness were prepared using a cryostat and adhered to Superfrost Plus Slides (Fisher Scientific). Slides were stored at −20° C. until use. To visualize endogenous mCherry and mTurq2 fluorescence, slides were dried at room temperature for 15 minutes, rehydrated in PBS for 5 minutes, permeabilized with 1% Triton X-100 in PBS for 15 minutes, and counterstained with Alexa Fluor 488 Phalloidin (ThermoFisher Scientific) diluted 1:500 in blocking buffer (3% bovine serum albumin and 0.3% Triton X-100 in PBS). To immunostain for endogenous proteins, slides were dried at room temperature for 4-24 hours and rehydrated in PBS for 5 minutes. Antigen retrieval was then performed by pressure cooking slides in sodium citrate buffer (10 mM tri-sodium citrate dihydrate, 0.05% Tween-20, pH 6.0) for 20 minutes in an Instant Pot (Amazon). Slides were rinsed in PBS for 5 minutes, dried briefly, and sections outlined with an ImmEdge hydrophobic pen (Vector Laboratories). Sections were permeabilized with 1% Triton X-100 in PBS for 15 minutes and blocked with blocking buffer for one hour. Sections were then incubated in primary antibodies diluted in blocking buffer at room temperature for 12-24 hours. Sections were washed with blocking buffer three times for 10 minutes each. Sections were then incubated in AlexaFluor secondary antibodies (ThermoFisher Scientific) diluted 1:1,000 in blocking buffer at room temperature for 1-2 hours. In some cases, 5 μg/mL Hoechst 33342 (ThermoFisher Scientific) was added to the secondary antibody solution. Sections were washed with blocking buffer twice for 10 minutes each followed by one wash with PBS for 5 minutes. Slides were then mounted in ProLong Gold Antifade reagent (ThermoFisher Scientific).

The following primary antibodies were used: Cas9 (1:200, clone 7A9-3A3, Abcam ab191468), asialoglycoprotein receptor 1 (ASGR1) (1:500, clone 114, Sino Biological 50083-R114), mCherry (1:500, clone 16D7, ThermoFisher Scientific M11217), monoamine oxidase B (MAO-B) (1:1,000, Novus Biologicals NBP1-87493), lamin B2 (1:1,000, clone EPR9701(B), Abcam ab151735), actin (1:250, clone AC-74, Sigma Aldrich A2228), CD45 (1:500, Abcam ab10558), and Ki-67 (1:200, clone SP6, Abcam ab16667). The Cas9 antibody was directly conjugated to AlexaFluor 647 using the AlexaFluor 647 Antibody Labeling Kit (ThermoFisher Scientific). The actin antibody was directly conjugated to DyLight 405 using the DyLight 405 antibody labeling kit (ThermoFisher scientific).

Image Analysis

Images were acquired using a CSU-22 spinning disc confocal head (Yokogawa) with Borealis modification (Andor) mounted on an Axiovert 200M microscope (Zeiss) with 10× or 40× objectives (Zeiss), an Orca-ER CCD camera (Hamamatsu), and MetaMorph acquisition software (Molecular Devices).

Images were analyzed using Volocity (Quorum Technologies). To measure MAO-B and lamin B2 intensity, a single Z plane at the center of the cell was identified and the cytoplasm or nucleus was outlined to measure the signal intensity per μm. A similar procedure was done on sections stained only with secondary antibodies to calculate the average background intensity. This average background intensity was subtracted from each MAO-B and lamin B2 intensity measurement and the background-subtracted measurements were then normalized within a given sample (mCherry-positive or -negative hepatocytes within a single liver).

Immunoblotting

To prepare protein lysates from cultured cells, 500,000 cells were pelleted and resuspended in 100 μL of 2× Laemmli sample buffer (100 mM Tris pH 6.8, 20% glycerol, 4% SDS, 0.02% bromophenol blue, 5% β-mercaptoethanol). Lysates were homogenized by pipetting followed by trituration through a 31-gauge needle and boiled for 5 minutes. Samples were separated on homemade polyacrylamide gels and transferred to Immobilon-FL membranes (Millipore) via wet transfer. Membranes were blocked in 5% bovine serum albumin in TBST (50 mM Tris pH 8.0, 150 mM NaCl, 0.1% Tween-20) for 1 hour at room temperature. Membranes were incubated in primary antibody diluted in blocking solution at 4° C. with rocking overnight and washed with TBST for five minutes five times. Membranes were incubated in HRP-conjugated goat anti-rabbit secondary antibody (Abcam ab205718) diluted 1:50,000 in blocking solution at room temperature with rocking for one hour and washed with TBST for five minutes five times. Membranes were incubated in ECL Prime Western Blotting Detection Reagent (GE Healthcare) for five minutes and exposed to CL-Xposure Film (Thermo Fisher Scientific).

The following primary antibodies were used: Cxorf38 (1:1,000, Invitrogen PAS-62139) and Tap1 (1:1,000, Cell Signaling Technology 12341).

RNA Sequencing

For surgical resection time points, partial hepatectomies were performed on 8 week-old mice as previously described (41). For toxic injury time points, 8 week-old mice were injected intraperitoneally with 2 μL/gram of 25% carbon tetrachloride diluted in corn oil (Sigma Aldrich). For all time points, livers from three male C57BL/6J mice were harvested, flushed with PBS, immediately immersed in RNAlater (Qiagen), incubated at room temperature for 24 hours, and stored at −20° C. until future use. To isolate RNA, 30 mg of each tissue was removed from RNAlater and homogenized in 700 μL of QIAzol lysis reagent (Qiagen) using the TissueRuptor homogenizer (Qiagen). RNA was purified using the miRNeasy Kit (Qiagen) according to kit instructions and eluted in 30 μL of nuclease-free water. RNA sequencing libraries were prepared using KAPA mRNA HyperPrep Kit (KAPA Biosystems) according to manufacturer instructions. Briefly, 0.1-1 ug of total RNA was enriched for polyadenylated sequences using oligo-dT magnetic bead capture. The enriched mRNA fraction was then fragmented and first-strand cDNA generated using random primers. Strand specificity was achieved during second-strand cDNA synthesis by replacing dTTP with dUTP to quench the second strand during amplification. The resulting cDNA was A-tailed and ligated with indexed adapters. The library was amplified using a DNA polymerase that cannot incorporate past dUTPs to quench the second strand during PCR. The libraries were quantified using a KAPA qPCR Library Quantification Kit (KAPA Biosystems) as per manufacturer instructions. The samples were sequenced on a HiSeq 2500 (Illumina) based on qPCR concentrations. Base calls were performed by the instrument control software and further processed using the Offline Base Caller version 1.9.4 (Illumina). Samples were mapped with STAR version 2.6.1a (42) to the mouse genome release mm10, using a gtf file from ENSEMBL version GRCm38.91, and setting the maximum intron length (“alignIntronMax”) parameter to 50000. We ran featureCounts version 1.6 (43) to assign reads to genes using the same gft file and setting “−s” parameter to 2. We normalized gene counts with DESeq2 version 1.22.2 (44). FPKMs were calculated using the function fpkm within the DESeq2 package. The FPKMs values for the three replicates were averaged, and protein coding genes were selected based on the annotation in the gtf file.

sgRNA Library Preparation

Genes with an average FPKM>0.3 in any of the RNA sequencing time points were chosen to build a liver transcriptome-wide library. sgRNA sequences were designed using the Broad Institute GPP sgRNA Designer (12, 45) using the Azimuth 2.0 rule set. For genes which were not identified by the program, alternative gene names from ENSEMBL versions GRCm38.76-.93 were attempted. A small number of designed sgRNAs targeted multiple genes; the sgRNA names and gene names were manually annotated to indicate all targeted genes for these cases. Non-targeting and control-gene-targeting sgRNAs from Doench et al. 2014 (12) were also included. sgRNA sequences from this control set that were identical to a sequence already in our library were annotated according to the targeted gene; those that did not overlap with sequences in our sgRNA library were annotated as control sgRNAs. The library contains 71,878 sgRNAs targeting 13,189 genes.

For sgRNAs beginning with a nucleotide other than G, a G was prepended. The following adapters were added to all sgRNA sequences:

Upstream:
(SEQ ID NO: 25)
5′-TATCTTGTGGAAAGGACGAAACACC-3′
Downstream:
(SEQ ID NO: 26)
5′-AAGAGCTATGCTGGAAACAGCATAGC-3′

Multiple rounds of cloning were combined to generate the final plasmid library. The oligonucleotide library (Agilent Technologies) was amplified for 16 cycles using Q5 HotStart Polymerase (New England Biolabs) using a gradient annealing temperature ranging from 50-62° C. across 8, 50 μL reactions. Reactions were pooled and purified by DNA Clean and Concentrator 5 (Zymo Research). pLCv2-opti-stuffer-mCherry was digested as described (40), and either gel purified using a Zymoclean Gel DNA Recovery Kit (Zymo Research) followed by Ampure XP bead purification (Beckman Coulter) or DNA Clean and Concentrator 5. The library was assembled using NEBuilder HiFi DNA Assembly Master Mix (New England Biolabs) in 4×20 μL reactions at 50° C. for 1 hour using 100 ng of vector per 5-10 ng of PCR amplicon. The reactions were combined and 2.5 μL of the assembly reaction or a control reaction without amplicon were used to transform NEBS-alpha cells (New England Biolabs) to measure background assembly. Subsequently, the assembly reactions were combined, concentrated using Ampure XP beads, resuspended in 8 μL water, and used to electroporate 1-4 tubes of Endura DUO electrocompetent cells (Lucigen) at 1.8 kV distributed over 2 cuvettes (0.1 cm gap width) per tube using a Micropulser Electroporator (Bio-Rad Laboratories). 10-fold serial dilutions of a 10 μL aliquot were plated on LB plates with ampicillin at 100 μg/mL to assess electroporation efficiency, and the remainder of each electroporation (2 cuvettes) was plated on LB agar supplemented with 100 μg/mL ampicillin in 4×245 mm square bioassay dishes (Corning). Plates were incubated overnight at 30° C. and colonies were scraped the next morning. DNA was isolated using the ZymoPURE II Plasmid Maxiprep Kit (Zymo Research). Plasmid DNA from multiple rounds of assembly and electroporation were combined according to the measured electroporation efficiency to achieve 25-fold coverage of the library. sgRNA representation was measured by high-throughput sequencing as described below.

To improve coverage of some of the sgRNAs in the library, a second library containing ˜7,500 sgRNAs was synthesized and cloned as above, with the following modifications: assembly was performed using NEB Gibson Assembly mix (New England Biolabs) using a ratio of 200 ng vector:10 ng sgRNA in each 20 μL reaction, and the final combined and concentrated reaction was used to electroporate a single tube of Endura DUO cells.

Subsequent propagation of the plasmid library was performed using 50 ng plasmid library per single tube of Endura DUO cells.

All steps were performed according to manufacturer's instructions, except where noted.

Genomic DNA Isolation

Livers were harvested from mice, separated into individual lobes, minced into 15 mg pieces using a razor blade, snap-frozen in liquid nitrogen, and stored at −80° C. until use. Genomic DNA (gDNA) was isolated from livers using the illustra blood genomic Prep Mini Spin Kit (Cytiva) using one column for every 7.5 mg of tissue. The manufacturer's protocol was used with the following modifications: 20 μL of 10 mg/mL Proteinase K (Millipore-Sigma) solution in water was added per 7.5 mg of tissue. Tissue was disrupted by thoroughly pipetting prior to adding lysis buffer, vortexing, and incubating at 56° C. overnight. Elution was performed using 25 μL of water pre-heated to 70° C. Samples were combined by lobe and concentration was measured using the Qubit dsDNA HS Assay Kit (Invitrogen).

For the induction, equal amounts of gDNA from each lobe were combined within each mouse, and equal inputs from four mice were combined to prepare a single sequencing library. For the endpoint, gDNA from each lobe within a mouse was combined proportionally to the average lobe mass across mice measured at liver harvest. A sequencing library was prepared for each mouse individually using equal total gDNA input per mouse.

Sequencing Library Preparation and DNA Sequencing

All PCR reactions were performed in 50 μL reactions using ExTaq Polymerase (Takara Bio) with the following program:

| | | | | | |
|---|---|---|---|---|---|
| 1 | cycle | 95° | C. | 5 | min |
| 14 or 28 | cycles | 95° | C. | 10 | sec |
| | | 60° | C. | 15 | sec |
| | | 72° | C. | 45 | sec |
| 1 | cycle | 72° | C. | 5 | min |

| | | | | |
|---|---|---|---|---|
| 1 | cycle | 4° | C. | hold |

Using the following primers:

Forward:
(SEQ ID NO: 27)
AATGATACGGCGACCACCGAGATCTACACCGACTCGGTGCCACTTTT
Reverse:
(SEQ ID NO: 28)
CAAGCAGAAGACGGCATACGAGATCnnnnnnTTTCTTGGGTAGTTTGCAG
TTTT

Where “nnnnnn” denotes the barcode used for multiplexing.

10 ng of plasmid DNA was amplified for 14 cycles in 4×50 μL reactions. 1, 3, or 6 μg of gDNA was initially amplified for 28 cycles in 50 μL test PCR reactions. Subsequently, 226 μg of gDNA (induction) was used in 38 reactions, or 75 μg of gDNA (endpoint) was used in 25 reactions per mouse. All reactions were cleaned and concentrated using Ampure XP beads prior to sequencing for 50 cycles on an Illumina Hiseq 2500 using the following primers:

Read 1 sequencing primer:
(SEQ ID NO: 29)
GTTGATAACGGACTAGCCTTATTTAAACTTGCTATGCTGTTTCCAGCATA
GCTCTTAAAC
Index sequencing primer:
(SEQ ID NO: 30)
TTTCAAGTTACGGTAAGCATATGATAGTCCATTTTAAAACATAATTTTAA
AACTGCAAACTACCCAAGAAA

Base calls were performed by the instrument control software and further processed using the Offline Base Caller (Illumina) v. 1.9.4.

Screen Analysis

For initial measurement of sgRNA representation in the plasmid library or induction time point, sequencing reads were mapped to the library, each sgRNA was given a pseudocount of 1, and reads per million (RPM) was calculated as previously described (11). Raw counts were processed using MAGeCK for downstream analysis (13). The plasmid library and induction timepoint were used as control samples and to estimate variance, and each endpoint mouse was processed separately. For mouse 4, sgLmnb2_1 was removed prior to MAGeCK analysis, as the high representation of this sgRNA (an sgRNA used for development of the screening method) was likely due to contamination during sequencing library preparation. Counts data from Shohat et al. (day 18) and Tzelepis et al. (day 14) were processed individually using MAGeCK (22, 23). The corresponding plasmid libraries were used as control samples. For our screen, Shohat et al., and Tzelepis et al., the size factor for normalization was estimated using the control sgRNA set. For Tzelepis et al., the three replicate day 14 samples were processed together to generate a single gene score, and those three samples were used to estimate variance. For all screens, the gene test FDR threshold was set to 0.05, the sgRNA p-value was FDR-adjusted, and the gene score was calculated using the median. Twenty-four human hepatocellular carcinoma screens from the CRISPR (Avana) Public 20Q4 release were downloaded from the Broad DepMap portal using “Liver” as a lineage filter and “Hepatocellular Carcinoma” as a lineage subtype filter (24, 25).

All downstream analyses were performed in R version 3.6.0, and all plots were generated in either base R, using the R corrplot package, or in GraphPad Prism Version 7.0d. For comparisons within our screen, the gene scores from individual mice were not normalized across mice, as each mouse serves as a replicate screen. The gene score for each gene across mice was tested against all gene scores using an unpaired two-sample Wilcoxon test. The p-values from this test were adjusted using the Benjamini-Hochberg (FDR) procedure. The median log2 fold change across mice was used as input for pre-ranked GSEA using the c2.cp.kegg.v7.1.symbols.gmt gene sets. For comparisons between screens from different sources, all screens from all the sources in the specific comparison were quantile normalized to one another using the preprocessCore R package prior to calculating the median log2 fold change within the screens from each source. This normalized median log2 fold change was subtracted from the normalized median log2 fold change of our screens to generate a differential score used as input for pre-ranked GSEA using the c2.cp.kegg.v7.1.symbols.gmt gene sets. For converting mouse gene symbols to human gene symbols, the Mouse_Gene_Symbol_Remapping_to_Human_Orthologs_MSigDB.v7.1.chip was used (note that this excludes genes that have multiple annotations in either human or mouse).

Pearson correlation was used to compare gene effects between mice. Spearman correlation was used to compare gene effects with liver mRNA expression and protein half-life in mouse cells (14). The TUSON dataset of predicted tumor suppressor genes was sorted by ascending FDR q-value and the top 50 genes present in the compared datasets were used (20). Distribution differences were tested and p-values were calculated using the Kolmogorov-Smirnov test. A one-sided test was used for gene sets for which a phenotype could be predicted (core essential genes, tumor suppressor genes, and control enriched and depleted genes); all other comparisons used a two-sided test.

For comparison of sex-specific fitness effects, only genes with an average of >2 sgRNAs detected across mice were considered. For sex-specific enriched genes, a median fold change (log2)>0.5 across two mice of a given sex and an absolute median fold change (log2) difference of >0.25 compared to the other sex was required. To identify true tumor-suppressor-like genes, a median fold change (log2)>−0.5 was required in mice of the other sex. For sex-specific depleted genes, a median fold change (log2) of <−0.5 across two mice of a given sex and an absolute median fold change (log2) difference of >0.75 compared to the other sex was required. To identify true sex-specific essential genes, a median fold change (log2)>−0.5 was required in mice of the other sex.

Software Information
Conda version 4.9.2
MAGeCK-RRA version 0.5.9.2
R version 3.6.0
   R packages:
   preprocessCore version 1.48.0
   corrplot version 0.84
GSEA version 4.1.0
   Mouse_Gene_Symbol_Remapping_to_Human_Orthologs_MSigDB. v7.1.chip
Human_Symbol_with_Remapping_MsigDB.v7.1.chip
c2.cp.kegg.v7.1.symbols.gmt
GraphPad Prism version 7.0d

1. A method for generating an in vivo library for genomic screening, comprising
a. providing a non-human mammal comprising cells in an organ or tissue having a sequence encoding a Cas protein or a functional portion thereof,
b. introducing a plurality of single guide RNAs (sgRNA) into the non-human mammal with a viral vector to obtain an in vivo library for genomic screening comprising cells of the tissue or organ, wherein each cell comprises a sequence encoding a single guide RNA of the plurality of single guide RNAs.

2. The method of claim 1, wherein the non-human mammal is a mouse or rat.

3. The method of claim 1, wherein the non-human mammal is neonate or embryo.

4. The method of claim 1, wherein the non-human mammal has a disease or condition, is predisposed to a disease or condition, or has a genetic abnormality.

5. The method of claim 1, wherein the organ or tissue is the liver or hepatic tissue.

6. The method of claim 1, wherein the cells are proliferating.

7. The method of claim 1, wherein the Cas protein or functional fragment thereof is selected from the group consisting of Cas9 or a catalytically inactive Cas protein fused to an effector domain.

8. (canceled)

9. The method of claim 1, wherein the Cas protein or functional fragment thereof comprises a detectable label.

10. The method of claim 1, wherein expression of the Cas protein or functional fragment thereof is under control of an inducible promoter and/or wherein expression of the plurality of sgRNAs are under control of an inducible promoter.

11. (canceled)

12. The method of claim 1, wherein the plurality of sgRNA target the expressed genes of the cells or a substantial portion thereof.

13. The method of claim 1, wherein an average of more than two sgRNA species target each expressed gene.

14.-15. (canceled)

16. The method of claim 1, wherein the plurality of sgRNA comprise one or more species targeting one or more control genes.

17. The method of claim 1, wherein the viral vector is introduced by injection into a vein.

18.-19. (canceled)

20. The method of 1, wherein the viral vector integrates a nucleotide sequence encoding the sgRNA into the genome of the cells, optionally wherein the viral vector is a lentiviral or retroviral vector.

21. (canceled)

22. The method of claim 1, wherein about 10%, 20%, 25%, 50%, 75%, or 90% of the cells in an organ or tissue comprise sgRNA.

23. A non-human mammal comprising the in vivo library generated by the method of claim 1.

24. A method of in vivo screening for genomic sites in cells in an organ or tissue associated with a change in phenotype, comprising
a. providing a non-human animal comprising an in vivo library of a population of cells in an organ or tissue having a sequence encoding a Cas protein or a functional portion thereof and a sequence encoding an sgRNA of a plurality of sgRNAs targeting the genomic sites,
b. providing conditions under which the Cas protein or functional portion thereof and the sgRNA contact and modify the genomic sites,
c. detecting one or more phenotypic changes in the population of cells and determining a genomic site associated with the phenotypic change by identifying a sgRNA associated with the phenotypic change and determining the sgRNA target genomic site.

25. The method of claim 24, wherein the genomic sites are expressed genes in the population of cells.

26. (canceled)

27. The method of claim 24, wherein the non-human mammal is a mouse or rat.

28.-29. (canceled)

30. The method of claim 24, wherein the organ or tissue is the liver or hepatic tissue.

31.-48. (canceled)


SEQUENCE LISTING (30 sequences)

SEQ ID NO: 1 - DNA, 36 bp, Artificial
  optimized scaffold of lentiCRISPRv2 derivative
gtttaagagctatgctggaaacagcatagcaagttt

SEQ ID NO: 2 - DNA, 1103 bp, Artificial
  HS-CRM8-TTRmin-dsRed
gaattcgctagcaccggcgcgccgggggaggctgctggtgaatattaaccaaggtcaccccagttatcggaggagcaaacaggggctaagtccacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgaccggttctagagcgctgccaccatggtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaaactgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaagactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgaaatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgaaggccgccaccacctgttcctgggatccggcgcaacaaacttctctctgctgaaacaagccggagatgtcgaagagaatcctggaccgaccgag

SEQ ID NO: 3 - DNA, 41 bp, Artificial
  primer
ggttctagagcgctgccaccatggtgagcaagggcgaggag

SEQ ID NO: 4 - DNA, 30 bp, Artificial
  primer
gccggatcccttgtacagctcgtccatgcc

SEQ ID NO: 5 - DNA, 26 bp, Artificial
  primer
tgaacgcgttaagtcgacaatcaacc

SEQ ID NO: 6 - DNA, 25 bp, Artificial
  primer
tcgaggctgatcagcgggtttaaac

SEQ ID NO: 7 - DNA, 24 bp, Artificial
  primer
caccggggccactagggacaggat

SEQ ID NO: 8 - DNA, 24 bp, Artificial
  sgAAVS1_R
aaacatcctgtccctagtggcccc

SEQ ID NO: 9 - DNA, 25 bp, Artificial
  sgMaob_1_F
caccgacggataaaggatatacttg

SEQ ID NO: 10 - DNA, 25 bp, Artificial
  sgMaob_1_R
aaaccaagtatatcctttatccgtc

SEQ ID NO: 11 - DNA, 25 bp, Artificial
  sgMaob_2_F
caccgggaaaatcatatgccttcag

SEQ ID NO: 12 - DNA, 25 bp, Artificial
  sgMaob_2_R
aaacctgaaggcatatgattttccc

SEQ ID NO: 13 - DNA, 25 bp, Artificial
  sgLmnb2_1_F
caccgaggtacgggagacccgacgg

SEQ ID NO: 14 - DNA, 25 bp, Artificial
  sgLmnb2_1_R
aaacccgtcgggtctcccgtacctc

SEQ ID NO: 15 - DNA, 25 bp, Artificial
  sgLmnb2_2_F
caccgctgcgcacctacctcaccgt

SEQ ID NO: 16 - DNA, 25 bp, Artificial
  sgLmnb2_2_R
aaacacggtgaggtaggtgcgcagc

SEQ ID NO: 17 - DNA, 25 bp, Artificial
  sgCxorf38_1_F
caccggtcaaccacaaagtgatcac

SEQ ID NO: 18 - DNA, 25 bp, Artificial
  sgCxorf38_1_R
aaacgtgatcactttgtggttgacc

SEQ ID NO: 19 - DNA, 25 bp, Artificial
  sgCxorf38_2_F
caccgtctgcgcatacctgacactg

SEQ ID NO: 20 - DNA, 25 bp, Artificial
  sgCxorf38_2_R
aaaccagtgtcaggtatgcgcagac

SEQ ID NO: 21 - DNA, 25 bp, Artificial
  sgTap1_1_F
caccgggtgccaacgagccactgag

SEQ ID NO: 22 - DNA, 25 bp, Artificial
  sgTap1_1_R
aaacctcagtggctcgttggcaccc

SEQ ID NO: 23 - DNA, 25 bp, Artificial
  sgTap1_2_F
caccgggtagagaacgaatgagaca

SEQ ID NO: 24 - DNA, 25 bp, Artificial
  sgTap1_2_R
aaactgtctcattcgttctctaccc

SEQ ID NO: 25 - DNA, 25 bp, Artificial
  Upstream sgRNA adapter
tatcttgtggaaaggacgaaacacc

SEQ ID NO: 26 - DNA, 26 bp, Artificial
  Downstream sgRNA adapter
aagagctatgctggaaacagcatagc

SEQ ID NO: 27 - DNA, 47 bp, Artificial
  primer
aatgatacggcgaccaccgagatctacaccgactcggtgccactttt

SEQ ID NO: 28 - DNA, 54 bp, Artificial
  primer
  misc_feature (26)..(31): n is a, c, g, or t
caagcagaagacggcatacgagatcnnnnnntttcttgggtagtttgcagtttt

SEQ ID NO: 29 - DNA, 60 bp, Artificial
  primer
gttgataacggactagccttatttaaacttgctatgctgtttccagcatagctcttaaac

SEQ ID NO: 30 - DNA, 71 bp, Artificial
  primer
tttcaagttacggtaagcatatgatagtccattttaaaacataattttaaaactgcaaactacccaagaaa